SEQUENCE LISTING (12 sequences)

SEQ ID NO: 1 - DNA, 25 bp, Artificial Sequence
  Synthetic Construct
caccgccattgttcaatatcgtccg

SEQ ID NO: 2 - DNA, 25 bp, Artificial Sequence
  Synthetic Construct
aaaccggacgatattgaacaatggc

SEQ ID NO: 3 - DNA, 19 bp, Artificial Sequence
  Synthetic Construct
tcaccaccatggagaaggc

SEQ ID NO: 4 - DNA, 20 bp, Artificial Sequence
  Synthetic Construct
gctaagcagttggtggtgca

SEQ ID NO: 5 - DNA, 23 bp, Artificial Sequence
  Synthetic Construct
tcttggtgaccctccggattcgg

SEQ ID NO: 6 - DNA, 21 bp, Artificial Sequence
  Synthetic Construct
tcagccaggtccacgggcaga

SEQ ID NO: 7 - DNA, 20 bp, Artificial Sequence
  Synthetic Construct
aggaaggggatggtaggaga

SEQ ID NO: 8 - DNA, 20 bp, Artificial Sequence
  Synthetic Construct
acacaagcacacatgcatca

SEQ ID NO: 9 - DNA, 24 bp, Artificial Sequence
  Synthetic Construct
ctgtggccttgtgtctgtgagtac

SEQ ID NO: 10 - DNA, 22 bp, Artificial Sequence
  Synthetic Construct
cctagcccaaggcaaggaggac

SEQ ID NO: 11 - DNA, 18 bp, Artificial Sequence
  Synthetic Construct
atgtagggaaaagcccgg

SEQ ID NO: 12 - DNA, 20 bp, Artificial Sequence
  Synthetic Construct
gaacatgtcccaacatgttg

(19) United States
(12) Patent Application Publication
(10) Pub. No.: US 2024/0307220 A1
(43) Pub. Date: Sep. 19, 2024

(54) Combination Therapy to Treat Temperature Sensitive Mutant Tumors

(71) Applicant: H. LEE MOFFITT CANCER CENTER AND RESEARCH INSTITUTE INC., Tampa, FL (US)

(72) Inventor: Jiandong CHEN, Tampa, FL (US)

(21) Appl. No.: 18/548,687

(22) PCT Filed: Mar. 3, 2022
(86) PCT No.: PCT/US2022/070938

§ 371 Date: Sep. 1, 2023

Related U.S. Application Data
(60) Provisional application No. 63/157,416, filed on Mar. 5, 2021.

(51) Int. Cl. A61F 7/12; A61F 7/00; A61F 7/10; A61K 45/06; G01N 33/68

(52) U.S. Cl. CPC A61F 7/12; A61F 7/10; A61K 45/06; G01N 33/6893; A61F 2007/0022; A61F 2007/0056; A61F 2007/0063; A61F 2007/0095; A61F 2007/126; G01N 2333/4748

(57) ABSTRACT

Disclosed herein is a method for treating a tumor having a temperature sensitive p53 (ts p53) mutation in a subject in need thereof. The method involves first administering to the subject a therapeutically effective amount of a chemotherapeutic drug, then inducing moderate hypothermia in the tumor for a duration sufficient to activate the mutant p53 to enhance the efficacy of the drug.

### CROSS-REFERENCE TO RELATED APPLICATIONS

This application claims benefit of U.S. Provisional Application No. 63/157,416, filed Mar. 5, 2021, which is hereby incorporated herein by reference in its entirety.

### STATEMENT REGARDING FEDERALLY SPONSORED RESEARCH OR DEVELOPMENT

This invention was made with Government Support under Grant Nos. CA141244, CA186917, GM115556, and CA260356 awarded by the National Institutes of Health. The Government has certain rights in the invention.

### SEQUENCE LISTING

This application contains a sequence listing filed in electronic form as an ASCII.txt file entitled “320803_2620_Sequence_Listing_ST25” created on Feb. 28, 2022, having 2,587 bytes. The content of the sequence listing is incorporated herein in its entirety.

### BACKGROUND

P53 is a transcription factor inducible by stress signals such as DNA damage, oncogene activation, and nutrient deprivation. P53 tetramer binds to a specific DNA sequence and activates genes involved in cell cycle, apoptosis, and energy metabolism (Vousden K H, et al. Nat Rev Mol Cell Biol 2007 8:275-83). P53 is the most frequently mutated gene in human cancer (>50% overall mutation rate). Most p53 mutations (˜80%) are amino acid substitutions in the DNA binding domain that cause misfolding or disrupt the DNA binding surface (Leroy B, et al. Hum Mutat 2014 35:672-88; Joerger A C, et al. Annu Rev Biochem 2016 85:375-404). As a result, mutant p53 does not bind DNA or activate target genes.

Mutant p53 is resistant to MDM2-mediated ubiquitination and accumulates to high levels in tumor cells (Peng Y, et al. J Biol Chem 2001 276:40583-90; Li D, et al. Molecular cancer research 2011 9:577-88). Restoring the DNA binding function of mutant p53 is an attractive strategy with significant therapeutic potential (Khoo K H, et al. Nature reviews Drug discovery 2014 13:217-36; Bullock A N, et al. Nat Rev Cancer 2001 1:68-76). However, currently there are no effective mutant p53-targeted drugs approved for clinical use. Wild type (wt) p53 has poor structural stability.

### SUMMARY

Disclosed herein is a method for treating a tumor having a temperature sensitive p53 (ts p53) mutation in a subject in need thereof. The method involves first administering to the subject a therapeutically effective amount of a chemotherapeutic drug, then induce moderate hypothermia in the tumor for a duration sufficient to activate the mutant p53, which enhances the efficacy of the chemotherapy drug.

Hypothermia can be induced in the tumor or subject by a variety of different means. In some embodiments, hypothermia is induced by administering to the subject an effective amount of an anti-psychotic drug, such as Zuclopenthixol, Flupenthixol, Chlorprothixen, Tiotixene, Clopenthixol, Thioridazine, Chlorpromazine, Levomepromazine, Cyamemazine, Periciazine, Pipothiazine, Fluphenazine, Trifluoperazine, Perphenazine, Prochlorperazine, Promazine, Mesoridazine, Haloperidol, Pipamperone, Droperidol, Benperidol, Tiapride, Sulpiride, Amisulpiride, Sultopride, Loxapine, Pimozide, Zotepine, Prothipendyl, Penfluridol, Risperidone, Clozapine, Olanzapine, Quetiapine, Aripiprazole, Ziprasidone, or any combination thereof.

In some embodiments, hypothermia is induced with core and/or peripheral cooling of the subject. In some embodiments, the tumor temperature is maintained at a temperature of 32° C. to 34° C. for at least 24, 25, 26, 27, 28, 29, 30 31, 32, 33, 34, 35, 36, or 48 hours.

The method can be used to treat any tumor with a temperature sensitive mutation. A previous study using a yeast transactivation assay at 30° C. identified 142 p53 ts mutations out of a library of 2,314 mutants covering all single nucleotide mutations in the DNA binding domain (Shiraishi K, et al. J Biol Chem 2004 279:348-55, which is incorporated by reference for the identification of these mutants).

In some embodiments, the ts p53 mutation is an amino acid substitution selected from the group consisting of S99, F113, Y126, N131, C135, A138, V143, W146, P151, P152, G154, T155, R156, R158, M160, A161, Q165, V172, R175, A189, P190, H193, R196, V197, Y205, D208, T211, F212, R213, H214, S215, V216, V217, P219, P223, C229, T231, I232, Y234, S240, M246, N247, R249, P250, I251, L252, T253, I254, I255, T256, L257, N268, F270, E271, V272, V274, R282, R283, E285, and E286, or any combination thereof.

In some embodiments, the ts p53 mutation is selected from the group consisting of S99F, F113C, Y126C, N131H, C135W, A138V, V143A, W146G, P151A, P152L, G154V, T155I, R156G, R158H, M160I, A161G, Q165L, V172F, R175G, A189P, P190T, H193Y, R196L, V197L, Y205N, D208A, T211A, F212V, R213G, H214R, S215I, V216L, V217G, P219L, P223A, C229G, T231A, I232F, Y234C, S240R, M246V, N247I, R249K, P250L, I251L, L252F, T253A, I254F, I255F, T256A, L257V, N268I, F270I, E271K, V272M, V274A, R282W, R283H, E285K, and E286G, or any combination thereof. In some embodiments, the ts p53 mutation is selected from the group consisting of S99F, A138V, V143A, P151A, P152L, P152T, G154V, T155I, M160I, A161G, V172F, R175G, R175L, H193R, V197L, Y205N, T211A, H214R, V216L, P219L, Y234C, Y234H, M246V, N247I, P250L, L252F, I254F, T256A, V272M, R282W, R283H, E285K, E286G, and E286K, or any combination thereof.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B show somatic p53 ts mutation frequency and distribution. FIG. 1A is a COSMIC plot of p53 missense mutations detected in cancer. FIG. 1B shows P53 ts mutations from Table 1 plotted in the same scale as FIG. 1A. Top-10 codons that generated the majority of ts mutants are marked.

FIGS. 2A to 2D show validation of p53 ts mutants in human cells. FIG. 1A shows P53 mutants transiently expressed using lentivirus vector in H1299 cells tested for activation of p21-luc reporter at 37° C., or after shifting to 32° C. for 18 hrs.

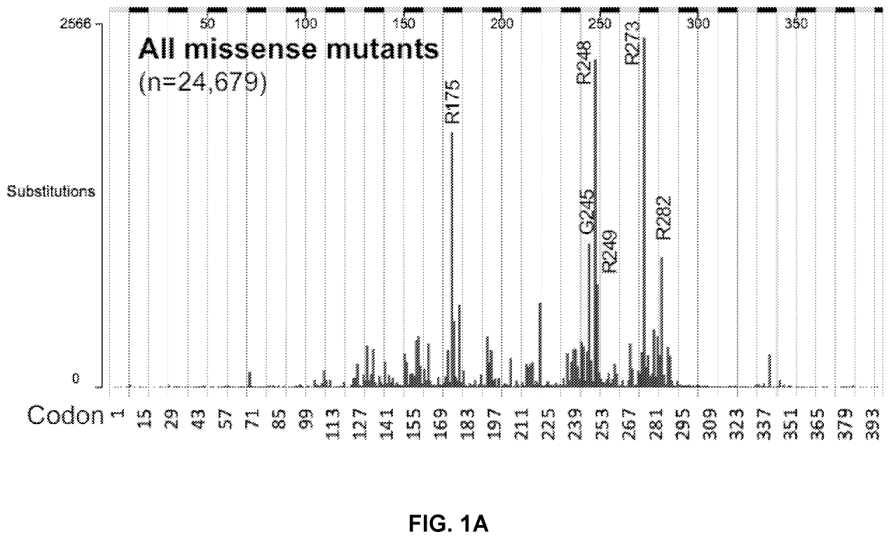

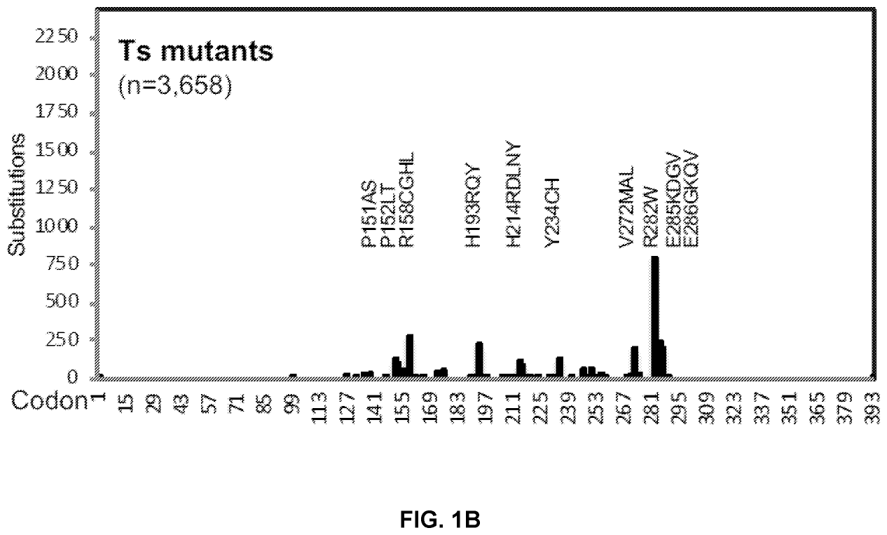

FIGS. 1B and 1C show representative p53 ts mutants stably expressed in H1299 cells tested for induction of target gene expression after 20 hrs at 32° C. CPT (0.5 μM for 20 hrs) was added when cells were shifted to 32° C. FIG. 1D shows H1299 cells stably expressing p53 mutants where treated with 0.5 μM CPT at 32° C. for 20 hrs. P53 binding to PUMA promoter was determined by ChIP. The results are average of 3 experiments (mean±SD).

FIGS. 3A to 3D show induction of growth arrest and cell death by p53 ts mutants. FIG. 3A shows H1299 cells stably infected with lentivirus expressing ts p53 mutants were shifted from 37° C. to 32° C. for 48 hrs and labeled with 3H-thymidine for 3 hrs. DNA replication rate was measured by scintillation counting and normalized to cell number. FIG. 3B shows H1299 stably expressing ts p53 mutants and non-ts R175H control were treated with 0.5 μM CPT for 48 hrs at 37° C. or 32° C. in culture medium with 1% FBS. PARP cleavage was determined by Western blot. FIGS. 3C and 3D show H1299 stably expressing ts p53 mutants treated with CPT at indicated concentrations for 48 hrs at 37° C. or 32° C. Cell viability was determined by MTS assay. The results are average of 3 experiments (mean±SD).

FIGS. 4A to 4E show molecular chaperones mediate refolding of stockpiled ts p53 at permissive temperature. FIG. 4A shows H1299 expressing A138V was shifted from 37° C. to 32° C. for indicated durations, or pre-incubated at 32° C. for 16 hrs and shifted back to 37° C. for indicated durations. P53 binding to p21 promoter was determined by ChIP. FIG. 4B shows H1299 cells expressing A138V was shifted to 32° C. in the absence or presence of 100 pg/ml cycloheximide (CHX) or 50 μM 17-AAG for indicated durations. P53 DNA binding was determined by ChIP. FIG. 4C is a diagram of cell-free luciferase fragment complementation assay that detects the DNA binding of p53-Cluc fusion protein. ZF-Nluc and p53-Cluc binding to DNA containing ZF and p53 sites juxtapose Nluc and Cluc domains to restore luciferase activity. FIG. 4D shows P53-Cluc and R282W-Cluc pre-treated for 30 min at 34° C. to inactivate R282W. The heat-treated p53-Cluc proteins were mixed with ZF-Nluc, DNA, ATP, E. coli extract containing 5 chaperones and incubated for 3 hrs at 23° C. P53 DNA binding was detected by measuring luciferase activity. The results are average of 3 experiments (mean±SD). **p<0.01. FIG. 4E shows coomassie staining of E. coli extract expressing molecular chaperones.

FIGS. 5A to 5C show activation of endogenous ts p53 mutants in tumor cell lines. FIG. 5A shows cell lines with endogenous ts mutant p53 (non-ts mutant alleles marked by *) cultured at 37° C. or shifted to 32° C. for 24 hrs and analyzed by Western blot. CPT was added to 0.5 μM where indicated. FIG. 5B shows GA10 cells infected with lentivirus expressing Cas9 and p53 gRNA. A clonal cell line without p53 was analyzed by Western blot after culturing at 37° C. or 32° C. for 24 hrs. FIG. 5C shows GA10 cells with and without p53 knockout seeded at identical starting numbers and cultured at 37° C. or 32° C. Cell numbers were counted daily and plotted. The results are average of 3 experiments (mean±SD).

FIGS. 6A to 6D show hypothermia inhibits the growth of tumors expressing ts p53. FIG. 6A shows nude mice injected with CHA (N6-cyclohexyladenine) and kept in a 28° C. environment to maintain body temperature at 32° C. FIG. 6B shows nude mice inoculated subcutaneously with GA10 cells and tumors allowed to grow to approximately 200 mm³. One group of mice was treated with 32° C. hypothermia in 24 hr×6 format. Control animals (37° C.) were not treated. FIG. 6C shows GA10 and GA10-p53^{KO} tumors treated with 32° C. hypothermia in 24 hr×6 format and tumor growth was monitored. FIG. 6D shows H1963 and H1963-p53^{KO} subcutaneous tumors treated with 32° C. hypothermia in 24 hr×6 format and tumor growth was monitored. Tumor size (mean±SD) was plotted over time.

FIGS. 7A to 7E show hypothermia cooperates with chemotherapy to induce regression of tumors expressing ts p53. FIG. 7A shows nude mice with subcutaneous GA10 and GA10-p53^{KO} tumors treated with combination of CPT and 32° C. hypothermia in 32 hr×5 format. CPT (1.5 mg/kg) was given at the beginning of each hypothermia cycle. Average size of tumors was plotted over time (mean±SD). FIG. 7B contains pictures of a GA10 partial response tumor before and after treatment. FIG. 7C is a waterfall plot showing combined results of 3 experiments. Tumors with durable remission are marked as “cured”. FIG. 7D contains individual growth curves of representative GA10 tumors from FIG. 7A that achieved durable remission after treatment. FIG. 7E contains individual growth curves of GA10 tumors from FIG. 7A that relapsed and received re-treatment.

FIGS. 8A to 8D show R282W is temperature-sensitive in human cells. FIG. 8A shows H1299 cells transfected with p53-responsive BP100-luc reporter and p53 hotspot mutants. The cells were cultured at 32° C. for 24 hrs and activation of the reporter was determined. The results were average of 3 experiments. FIG. 8B shows H1299 cells transfected with PUMA promoter-luc reporter and p53 mutants. The cells were cultured at 37° C. or 32° C. for 24 hrs and activation of the reporter was determined. FIG. 8C shows H1299 cells stably transfected with indicated plasmids at 37° C. Cells were cultured at 34° C. or 32° C. for 20 hrs and p53 target gene expression was analyzed by Western blot. FIG. 8D shows H1299 cells expressing p53 mutants kept at 37° C. or shifted to 32° C. for 18 hrs. P53 from identical amount of extract was immunoprecipitated with wt conformation-specific Pab1620 or mutant conformation-specific Pab240 antibodies and detected by Western blot using pan-specific antibody FL393. R175H was used as non-ts control.

FIGS. 9A to 9C show activation of reporter genes by frequently observed ts p53 mutants. H1299 cells were transiently transfected with p53 mutants and indicated luciferase reporters for 24 hrs at 37° C. The cells were kept at 37° C. or shifted to 32° C. for 18 hrs and analyzed for luciferase activity. The results are average of 3 experiments (mean±SD).

FIGS. 10A to 10F show induction of endogenous targets by ts p53 mutants. P53 ts mutants were stably expressed in H1299 cells using lentivirus vector. Cells were tested for induction of target gene expression after 20 hrs at 32° C. by Western blot. CPT (0.5 μM for 20 hrs) was added when cells were shifted to 32° C. A138V was used as a benchmark to facilitate comparison between gels.

FIGS. 11A and 11B show kinetics of ts p53 activation and inactivation by temperature shift. FIG. 11A shows H1299 cells expressing ts p53 mutants kept at 37° C. or shifted to 32° C. for indicated durations. P53 activation was analyzed by Western blot. FIG. 11B shows H1299 cells expressing ts p53 mutants kept at 32° C. for 24 hrs to activate p53, followed by shifting to 37° C. for indicated durations and analyzed by Western blot for down regulation of p53 activity.

FIGS. 12A to 12C show effect of 32° C. culture on p53 activity. FIG. 12A shows cell lines with different p53 status [Wt, ts (A138V), non-ts (R280K)] cultured at 32° C. for 20 hrs, or treated with 10 Gy gamma radiation for 4 hrs. Western blot was performed to determine the induction of p53 target genes. FIGS. 12B and 12C show endogenous ts p53 in the cell lines knocked out using lentivirus expressing Cas9 and p53 gRNA. Clonal cell lines without p53 were analyzed by Western blot after culturing at 37° C. or 32° C. for 18 hrs.

FIGS. 13A to 13E show tumors cells with ts p53 undergo cell cycle arrest and apoptosis at 32° C. FIG. 13A shows cells with endogenous ts p53 mutants were shifted from 37° C. to 32° C. for 24 hrs and labeled with ³H-thymidine for 3 hrs. DNA replication rate was measured by scintillation counting. H1299 and H1299 with stable expression of A138V were used as controls. FIGS. 13B and 13C show GA10 and GA10-p53^{KO} cells shifted to 32° C. for 48 hrs. Apoptotic cells were identified by Annexin V/7-AAD double staining and FACS analysis and compared to 37° C. controls. FIG. 13D shows quantification of apoptosis in FIGS. 13B and 13C. FIG. 13E shows ARF mRNA levels in the indicated cell lines determined using qRT-PCR. The results represent 3 experiments (mean±SD). **p<0.01.

FIGS. 14A to 14D show effect of low-dose CPT treatment alone on tumor growth. FIG. 14A shows nude mice inoculated subcutaneously with GA10 and GA10-p53^{KO} cells. Tumor growth at 37° C. (without hypothermia treatment) was determined at indicated time points. FIG. 14B shows nude mice inoculated subcutaneously with H1963 and H1963-p53^{KO} cells. Tumor growth at 37° C. (without hypothermia treatment) was determined at indicated time points. FIG. 14C shows nude mice inoculated subcutaneously with GA10 cells. When tumors reached approximately 100 mm³ the mice were injected i.p. with 1.5 mg/kg CPT every 3 days. Tumor growth was monitored over time (mean±SD). FIG. 14D shows nude mice inoculated subcutaneously with GA10 and GA10-p53KO tumors treated with 1.5 mg/kg CPT at indicated time points without hypothermia. Average size of tumors was plotted over time.

FIGS. 15A and 15B show effects of hypothermia and CPT combination on tumors. FIG. 15A shows individual growth curves of GA10-p53^{KO} tumors treated with hypothermia+CPT combination, relapsed, and retreated. FIG. 15B shows Western blot of tumors (n=3) with and without 24 hr hypothermia+CPT treatment.

FIG. 16 shows induction of tumor apoptosis by combination of hypothermia and chemotherapy. Nude mice with tumors were treated for 24 hrs with 32° C. hypothermia and 1.5 mg/kg CPT combination. Tumors were harvested and sections were subjected to TUNEL staining to detect apoptotic cells.

FIGS. 17A and 17B show hypothermia treatment given after chemotherapy enhances drug response in bladder cancer. FIG. 17A shows analysis of bladder cancer cell line response to temperature shift. Three cell lines expressing endogenous TS p53 mutants showed induction of p53 targets MDM2 and p21 at 32° C. FIG. 17B shows TS cell line UC3 was incubated with drugs for 2 hrs at 37° C., and washed to remove the drugs. The washed cells were incubated at 32° C. or 37° C. for 24 hrs, followed by 7 days at 37° C. and crystal violet staining for colony formation.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an agent can be an oligomer of nucleic acids, amino acids, or carbohydrates including, but not limited to proteins, peptides, oligonucleotides, ribozymes, DNAzymes, glycoproteins, RNAi agents (e.g., siRNAs), lipoproteins, aptamers, and modifications and combinations thereof. In some embodiments, an active agent is a nucleic acid, e.g., miRNA or a derivative or variant thereof.

Chemotherapeutics Non-limiting examples of known cancer drugs includes Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alpelisib, Alunbrig (Brigatinib), Ameluz (Aminolevulinic Acid Hydrochloride), Amifostine, Aminolevulinic Acid Hydrochloride, Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mknl), Atezolizumab, Avapritinib, Avastin (Bevacizumab), Avelumab, Axicabtagene Ciloleucel, Axitinib, Ayvakit (Avapritinib), Azacitidine, Azedra (Iobenguane 1131), Balversa (Erdafitinib), Bavencio (Avelumab), BEACOPP, Belantamab Mafodotin-blmf, Beleodaq (Belinostat), Belinostat, Bendamustine Hydrochloride, Bendeka (Bendamustine Hydrochloride), BEP, Besponsa (Inotuzumab Ozogamicin), Bevacizumab, Bexarotene, Bicalutamide, BiCNU (Carmustine), Binimetinib, Blenrep (Belantamab Mafodotin-blmf), Bleomycin Sulfate, Blinatumomab, Blincyto (Blinatumomab), Bortezomib, Bosulif (Bosutinib), Bosutinib, Braftovi (Encorafenib), Brentuximab Vedotin, Brexucabtagene Autoleucel, Breyanzi (Lisocabtagene Maraleucel), Brigatinib, Brukinsa (Zanubrutinib), BuMel, Busulfan, Busulfex (Busulfan), Cabazitaxel, Cablivi (Caplacizumab-yhdp), Cabometyx (Cabozantinib-S-Malate), Cabozantinib-S-Malate, CAF, Calaspargase Pegol-mknl, Calquence (Acalabrutinib), Campath (Alemtuzumab), Camptosar (Irinotecan Hydrochloride), Capecitabine, Caplacizumab-yhdp, Capmatinib Hydrochloride, CAPOX, Carac (Fluorouracil—Topical), Carboplatin, CARBOPLATIN-TAXOL, Carfilzomib, Carmustine, Carmustine Implant, Casodex (Bicalutamide), CEM, Cemiplimab-rwlc, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar (Clofarabine), CMF, Cobimetinib Fumarate, Cometriq (Cabozantinib-S-Malate), Copanlisib Hydrochloride, COPDAC, Copiktra (Duvelisib), COPP, COPP-ABV, Cosmegen (Dactinomycin), Cotellic (Cobimetinib Fumarate), Crizotinib, CVP, Cyclophosphamide, Cyramza (Ramucirumab), Cytarabine, Dabrafenib Mesylate, Dacarbazine, Dacogen (Decitabine), Dacomitinib, Dactinomycin, Danyelza (Naxitamab-gqgk), Daratumumab, Daratumumab and Hyaluronidase-fihj, Darbepoetin Alfa, Darolutamide, Darzalex (Daratumumab), Darzalex Faspro (Daratumumab and Hyaluronidase-fihj), Dasatinib, Daunorubicin Hydrochloride, Daunorubicin Hydrochloride and Cytarabine Liposome, Daurismo (Glasdegib Maleate), Decitabine, Decitabine and Cedazuridine, Defibrotide Sodium, Defitelio (Defibrotide Sodium), Degarelix, Denileukin Diftitox, Denosumab, Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Durvalumab, Duvelisib, Efudex (Fluorouracil—Topical), Eligard (Leuprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-lzsg, Emend (Aprepitant), Empliciti (Elotuzumab), Enasidenib Mesylate, Encorafenib, Enfortumab Vedotin-ejfv, Enhertu (Fam-Trastuzumab Deruxtecan-nxki), Entrectinib, Enzalutamide, Epirubicin Hydrochloride, EPOCH, Epoetin Alfa, Epogen (Epoetin Alfa), Erbitux (Cetuximab), Erdafitinib, Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Etoposide Phosphate, Everolimus, Evista (Raloxifene Hydrochloride), Evomela (Melphalan Hydrochloride), Exemestane, 5-FU (Fluorouracil Injection), 5-FU (Fluorouracil—Topical), Fam-Trastuzumab Deruxtecan-nxki, Fareston (Toremifene), Farydak (Panobinostat Lactate), Faslodex (Fulvestrant), FEC, Fedratinib Hydrochloride, Femara (Letrozole), Filgrastim, Firmagon (Degarelix), Fludarabine Phosphate, Fluoroplex (Fluorouracil—Topical), Fluorouracil Injection, Fluorouracil—Topical, Flutamide, FOLFIRI, FOLFIRI-BEVACIZUMAB, FOLFIRI-CETUXIMAB, FOLFIRINOX, FOLFOX, Folotyn (Pralatrexate), Fostamatinib Disodium, Fulphila (Pegfilgrastim), FU-LV, Fulvestrant, Gamifant (Emapalumab-lzsg), Gardasil (Recombinant HPV Quadrivalent Vaccine), Gardasil 9 (Recombinant HPV Nonavalent Vaccine), Gavreto (Pralsetinib), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Glasdegib Maleate, Gleevec (Imatinib Mesylate), Gliadel Wafer (Carmustine Implant), Glucarpidase, Goserelin Acetate, Granisetron, Granisetron Hydrochloride, Granix (Filgrastim), Halaven (Eribulin Mesylate), Hemangeol (Propranolol Hydrochloride), Herceptin Hylecta (Trastuzumab and Hyaluronidase-oysk), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin PFS (Idarubicin Hydrochloride), Idarubicin Hydrochloride, Idelalisib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imlygic (Talimogene Laherparepvec), Infugem (Gemcitabine Hydrochloride), Inlyta (Axitinib), Inotuzumab Ozogamicin, Inqovi (Decitabine and Cedazuridine), Inrebic (Fedratinib Hydrochloride), Interferon Alfa-2b, Recombinant, Interleukin-2 (Aldesleukin), Intron A (Recombinant Interferon Alfa-2b), lobenguane 1131, Ipilimumab, Iressa (Gefitinib), Irinotecan Hydrochloride, Irinotecan Hydrochloride Liposome, Isatuximab-irfc, Istodax (Romidepsin), Ivosidenib, Ixabepilone, Ixazomib Citrate, Ixempra (Ixabepilone), Jakafi (Ruxolitinib Phosphate), JEB, Jelmyto (Mitomycin), Jevtana (Cabazitaxel), Kadcyla (Ado-Trastuzumab Emtansine), Kepivance (Palifermin), Keytruda (Pembrolizumab), Kisqali (Ribociclib), Koselugo (Selumetinib Sulfate), Kymriah (Tisagenlecleucel), Kyprolis (Carfilzomib), Lanreotide Acetate, Lapatinib Ditosylate, Larotrectinib Sulfate, Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid Hydrochloride), Libtayo (Cemiplimab-rwlc), Lisocabtagene Maraleucel, Lomustine, Lonsurf (Trifluridine and Tipiracil Hydrochloride), Lorbrena (Lorlatinib), Lorlatinib, Lumoxiti (Moxetumomab Pasudotox-tdfk), Lupron Depot (Leuprolide Acetate), Lurbinectedin, Luspatercept-aamt, Lutathera (Lutetium Lu 177-Dotatate), Lutetium (Lu 177-Dotatate), Lynparza (Olaparib), Margenza (Margetuximab-cmkb), Margetuximab-cmkb, Marqibo (Vincristine Sulfate Liposome), Matulane (Procarbazine Hydrochloride), Mechlorethamine Hydrochloride, Megestrol Acetate, Mekinist (Trametinib Dimethyl Sulfoxide), Mektovi (Binimetinib), Melphalan, Melphalan Hydrochloride, Mercaptopurine, Mesnex (Mesna), Methotrexate Sodium, Methylnaltrexone Bromide, Midostaurin, Mitomycin, Mitoxantrone Hydrochloride, Mogamulizumab-kpkc, Monjuvi (Tafasitamab-cxix), Moxetumomab Pasudotox-tdfk, Mozobil (Plerixafor), MVAC, Mvasi (Bevacizumab), Myleran (Busulfan), Mylotarg (Gemtuzumab Ozogamicin), Nanoparticle Paclitaxel (Paclitaxel Albumin-stabilized Nanoparticle Formulation), Naxitamab-gqgk, Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nplate (Romiplostim), Nubeqa (Darolutamide), Nyvepria (Pegfilgrastim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Onureg (Azacitidine), Opdivo (Nivolumab), OPPA, Orgovyx (Relugolix), Osimertinib Mesylate, Oxaliplatin, Paclitaxel, Paclitaxel Albumin-stabilized Nanoparticle Formulation, PAD, Padcev (Enfortumab Vedotin-ejfv), Palbociclib, Palifermin, Palonosetron Hydrochloride, Palonosetron Hydrochloride and Netupitant, Pamidronate Disodium, Panitumumab, Panobinostat Lactate, Pazopanib Hydrochloride, PCV, PEB, Pegaspargase, Pegfilgrastim, Peginterferon Alfa-2b, PEG-Intron (Peginterferon Alfa-2b), Pemazyre (Pemigatinib), Pembrolizumab, Pemetrexed Disodium, Pemigatinib, Perjeta (Pertuzumab), Pertuzumab, Pertuzumab, Trastuzumab, and Hyaluronidase-zzxf, Pexidartinib Hydrochloride, Phesgo (Pertuzumab, Trastuzumab, and Hyaluronidase-zzxf), Piqray (Alpelisib), Plerixafor, Polatuzumab Vedotin-piiq, Polivy (Polatuzumab Vedotin-piiq), Pomalidomide, Pomalyst (Pomalidomide), Ponatinib Hydrochloride, Portrazza (Necitumumab), Poteligeo (Mogamulizumab-kpkc), Pralatrexate, Pralsetinib, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Qinlock (Ripretinib), Radium 223 Dichloride, Raloxifene Hydrochloride, Ramucirumab, Rasburicase, Ravulizumab-cwvz, Reblozyl (Luspatercept-aamt), R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), Relugolix, R-EPOCH, Retacrit (Epoetin Alfa), Retevmo (Selpercatinib), Revlimid (Lenalidomide), Ribociclib, R-ICE, Ripretinib, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rozlytrek (Entrectinib), Rubidomycin (Daunorubicin Hydrochloride), Rubraca (Rucaparib Camsylate), Rucaparib Camsylate, Ruxolitinib Phosphate, Rydapt (Midostaurin), Sacituzumab Govitecan-hziy, Sancuso (Granisetron), Sarclisa (Isatuximab-irfc), Sclerosol Intrapleural Aerosol (Talc), Selinexor, Selpercatinib, Selumetinib Sulfate, Siltuximab, Sipuleucel-T, Soltamox (Tamoxifen Citrate), Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), Tabrecta (Capmatinib Hydrochloride), TAC, Tafasitamab-cxix, Tafinlar (Dabrafenib Mesylate), Tagraxofusp-erzs, Tagrisso (Osimertinib Mesylate), Talazoparib Tosylate, Talc, Talimogene Laherparepvec, Talzenna (Talazoparib Tosylate), Tamoxifen Citrate, Tarceva (Erlotinib Hydrochloride), Targretin (Bexarotene), Tasigna (Nilotinib), Tavalisse (Fostamatinib Disodium), Taxotere (Docetaxel), Tazemetostat Hydrobromide, Tazverik (Tazemetostat Hydrobromide), Tecartus (Brexucabtagene Autoleucel), Tecentriq (Atezolizumab), Temodar (Temozolomide), Temozolomide, Temsirolimus, Tepadina (Thiotepa), Thalidomide, Thalomid (Thalidomide), Thioguanine, Thiotepa, Tibsovo (Ivosidenib), Tisagenlecleucel, Tocilizumab, Tolak (Fluorouracil—Topical), Topotecan Hydrochloride, Toremifene, Torisel (Temsirolimus), Totect (Dexrazoxane Hydrochloride), TPF, Trabectedin, Trametinib Dimethyl Sulfoxide, Trastuzumab, Trastuzumab and Hyaluronidase-oysk, Treanda (Bendamustine Hydrochloride), Trexall (Methotrexate Sodium), Trifluridine and Tipiracil Hydrochloride, Trisenox (Arsenic Trioxide), Trodelvy (Sacituzumab Govitecan-hziy), Truxima (Rituximab), Tucatinib, Tukysa (Tucatinib), Turalio (Pexidartinib Hydrochloride), Tykerb (Lapatinib Ditosylate), Ukoniq (Umbralisib Tosylate), Ultomiris (Ravulizumab-cwvz), Umbralisib Tosylate, Undencyca (Pegfilgrastim), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vidaza (Azacitidine), Vinblastine Sulfate, Vincristine Sulfate, Vincristine Sulfate Liposome, Vinorelbine Tartrate, VIP, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxeos (Daunorubicin Hydrochloride and Cytarabine Liposome), Xalkori (Crizotinib), Xatmep (Methotrexate Sodium), Xeloda (Capecitabine), XELIRI, XELOX, Xgeva (Denosumab), Xofigo (Radium 223 Dichloride), Xospata (Gilteritinib Fumarate), Xpovio (Selinexor), Xtandi (Enzalutamide), Yervoy (Ipilimumab), Yescarta (Axicabtagene Ciloleucel), Yondelis (Trabectedin), Yonsa (Abiraterone Acetate), Zaltrap (Ziv-Aflibercept), Zanubrutinib, Zarxio (Filgrastim), Zejula (Niraparib Tosylate Monohydrate), Zelboraf (Vemurafenib), Zepzelca (Lurbinectedin), Zevalin (lbritumomab Tiuxetan), Ziextenzo (Pegfilgrastim), Zinecard (Dexrazoxane Hydrochloride), Zirabev (Bevcizumab), Ziv-Aflibercept, Zofran (Ondansetron Hydrochloride), Zoladex (Goserelin Acetate), Zoledronic Acid, Zolinza (Vorinostat), Zometa (Zoledronic Acid), Zyclara (Imiquimod), Zydelig (Idelalisib), Zykadia (Ceritinib), and Zytiga (Abiraterone Acetate).

Hypothermia Disclosed herein are methods for treating certain cancers that involve combining chemotherapy and hypothermia. Methods for inducing hypothermia in a subject are known in the art and described, for example, in U.S. Pat. Nos. 6,736,837; 6,962,601; 6,983,749; US20020091426; US20170007445; Gavrielatos, G, et al. Ther Adv Cardiovasc Dis (2010) 4(5) 325-333; and Wion, D, et al. J Neurooncol (2017) 133:447-454, which are incorporated by reference in their entireties for the teaching of these methods.

In some embodiments, hypothermia is induced using core cooling using, for example, intravascular catheters (conduction), infusion of ice-cold fluids (conduction), extracorporeal circulation (conduction), or antipyretic agents. In some embodiments hypothermia is induced by peripheral cooling using, for example, fans (convection), air-circulating cooling blankets (convection), ice packs (conduction), water-circulating cooling blankets (conduction), immersion (conduction), specially designed beds (conduction), cooling caps (conduction), water and alcohol sprays (evaporation), sponge baths (evaporation), or exposure of skin (radiation).

In some embodiments, the hypothermia is mild hypothermia (e.g. 32-35° C.). In some embodiments, the hypothermia is moderate hypothermia (28-32° C.). In some embodiments, the hypothermia is not severe hypothermia (below 28° C.). In some embodiments, the hypothermia is 28° C., 29° C., 30° C., 31° C., 32° C., 33° C., 34° C., 35° C. In some embodiments, hypothermia is maintained for 12 to 48 hours, including 24 to 48 hours or 24 to 36 hours, such as 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours.

In some embodiments, intravesical hypothermia can be induced by circulating cooled buffer through the organ.

### Drug-Induced Hypothermia

Hypothermia can occur as a result of peripheral vasodilation, decrease in metabolic activity or exposure to cold environmental conditions. There are numerous drugs that affect body temperature including barbiturates, cyclic antidepressants, hypoglycemic agents, opioids, antihistamines, anticholinergics etc.

Therefore, in some embodiments, hypothermia is induced with a drug, such as an anti-psychotic drug, alone or in combination with core and/or peripheral cooling.

In some embodiments, hypothermia is induced the subject using an anti-psychotic drug. Certain anti-psychotic drugs, such as chlorpromazine, cause hypothermia as a side effect (Tarahovsky Y S, et al. Psychopharmacology (Berl) 2017 234:173-84; Kreuzer P, et al. J Clin Pharmacol 2012 52:1090-7).

Anti-psychotic drugs known to cause hypothermia include Zuclopenthixol, Flupenthixol, Chlorprothixen, Tiotixene, Clopenthixol, Thioridazine, Chlorpromazine, Levomepromazine, Cyamemazine, Periciazine, Pipothiazine, Fluphenazine, Trifluoperazine, Perphenazine, Prochlorperazine, Promazine, Mesoridazine, Haloperidol, Pipamperone, Droperidol, Benperidol, Tiapride, Sulpiride, Amisulpiride, Sultopride, Loxapine, Pimozide, Zotepine, Prothipendyl, Penfluridol, Risperidone, Clozapine, Olanzapine, Quetiapine, Aripiprazole, and Ziprasidone.

In some embodiment the anti-psychotic drug is a Butyrophenone, such as Benperidol, Bromperidol, Droperidol, Haloperidol, Moperone, Pipamperone, Timiperone, Melperone, or Lumateperone. In some embodiment the anti-psychotic drug is a Diphenylbutylpiperidine, such as Fluspirilene, Penfluridol, or Pimozide. In some embodiment the anti-psychotic drug is a Phenothiazine, such as Acepromazine, Chlorpromazine, Cyamemazine, Dixyrazine, Fluphenazine, Levomepromazine, Mesoridazine, Perazine, Pericyazine, Perphenazine, Pipotiazine, Prochlorperazine, Promazine, Promethazine, Prothipendyl, Thioproperazine, Thioridazine, Trifluoperazine, or Triflupromazine. In some embodiment the anti-psychotic drug is a Thioxanthene, such as Chlorprothixene, Clopenthixol, Flupentixol, Thiothixene, or Zuclopenthixol. In some embodiment the anti-psychotic drug is a Benzamide, such as Sulpiride, Sultopride, Veralipride, Amisulpride, Nemonapride, Remoxipride, or Sultopride. In some embodiment the anti-psychotic drug is a Tricyclic, such as Carpipramine, Clocapramine, Clorotepine, Clotiapine, Loxapine, Mosapramine, Asenapine, Clozapine, Olanzapine, Quetiapine, or Zotepine. In some embodiment the anti-psychotic drug is a Benzisoxazole, such as Iloperidone, Lurasidone, Paliperidone, Paliperidone palmitate, Perospirone, Risperidone, or Ziprasidone. In some embodiment the anti-psychotic drug is a Phenylpiperazine (quinolinone), such as Aripiprazole, Aripiprazole lauroxil, Brexpiprazole, or Cariprazine. In some embodiment the anti-psychotic drug is Blonanserin, Pimavanserin, or Sertindole.

In some embodiments, hypothermia is induced the subject using a tranquilizer or anesthetic. Mild hypothermia is extremely common during anesthesia and surgery. The basic process occurs as core body heat redistributes to the skin surface through anesthetic-induced vasodilation and depression of hypothalamic thermoregulatory centers. Heat loss occurs mostly through skin via radiation and convection. For example, midazolam slightly impairs thermoregulatory control, isoflurane and halothane impair thermoregulatory vasoconstriction, and propofol and volatile anesthetics inhibit nonshivering thermogenesis.

p53-Expressing Cancers

The cancer of the disclosed methods can be any cell in a subject undergoing unregulated growth, invasion, or metastasis. In some embodiments, the tumors have one or more temperature-sensitive p53 mutations.

In some aspects, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

In some cases, the cancer comprises a B and/or T cell acute lymphoblastic leukemia (ALL), Diffuse Large B cell Lymphoma (DLBCL), Follicular Lymphoma (FL), Marginal Zone Lymphoma (MZL), Chronic lymphocytic leukemia/Small lymphocytic lymphoma (CLL/SLL), Multiple myeloma (MM), Peripheral T cell lymphoma (PTCL), Cutaneous T cell lymphoma (CTCL), Burkitt Lymphoma, T cell lymphoma, or Multiple Myeloma.

### Embodiments

Embodiment 1. A method for treating a tumor having a temperature sensitive p53 (ts p53) mutation in a subject in need thereof, comprising
(a) inducing moderate hypothermia in the tumor for a duration sufficient to activate the mutant p53, and
(b) administering to the subject a chemotherapeutic drug.

Embodiment 2. The method of embodiment 1, wherein hypothermia is induced by administering to the subject an effective amount of an anti-psychotic drug.

Embodiment 3. The method of embodiment 2, wherein the anti-psychotic drug is selected from the group consisting of Zuclopenthixol, Flupenthixol, Chlorprothixen, Tiotixene, Clopenthixol, Thioridazine, Chlorpromazine, Levomepromazine, Cyamemazine, Periciazine, Pipothiazine, Fluphenazine, Trifluoperazine, Perphenazine, Prochlorperazine, Promazine, Mesoridazine, Haloperidol, Pipamperone, Droperidol, Benperidol, Tiapride, Sulpiride, Amisulpiride, Sultopride, Loxapine, Pimozide, Zotepine, Prothipendyl, Penfluridol, Risperidone, Clozapine, Olanzapine, Quetiapine, Aripiprazole, and Ziprasidone.

Embodiment 4. The method of embodiment 1, wherein hypothermia is induced with core and/or peripheral cooling of the subject.

Embodiment 5. The method of any one of embodiments 1 to 4, wherein the tumor temperature is maintained at a temperature of about 32° C. for 24 to 48 hours.

Embodiment 6. The method of any one of embodiments 1 to 5, further comprising assaying a sample from the subject for a ts p53 mutation.

Embodiment 7. The method of embodiment 6, wherein the ts p53 mutation is a S99F, A119V, Y126S, Y126D, K132N, K132R, M133T, A138V, T140Y, V143A, P152L, P151A, P152L, P152T, G154V, T155I, M160I/A161T, 1162F, T170R, V172F, R175K, R1751, R175P, R175Q, R175S, R175M, H179Q, E180K, R181G, R181H, H193R, V197L, Y205N, T211N, H214R, V216M, P219L, Y220C, Y220H, E224K, D228V, Y234C, Y234H, M237R, N239S, M246V, N247I, R248W, P250L, L252F, I254F, T256A, D259N, G266E, V272M, R273H, R273L, A276G, D281Y, R282W, R283H, E285K, E286G, E286K, or 286K/287D mutation, or any combination thereof.

Embodiment 8. The method of any one of embodiments 1 to 7, wherein the tumor is an intraperitoneal tumor.

Embodiment 9. The method of any one of embodiments 1 to 7, wherein the tumor is a bladder tumor.

Embodiment 10. The method of embodiment 9, wherein the bladder is cooled by circulating cooled buffer through a catheter in the bladder.

Embodiment 11. The method of any one of embodiments 1 to 7, wherein the moderate hypothermia is induced simultaneously with administration of the chemotherapeutic drug.

Embodiment 12. The method of any one of embodiments 1 to 7, wherein the moderate hypothermia is induced 1 minute to 1 day after the chemotherapeutic drug is administered.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Hypothermia Effectively Treats Tumors with Temperature-Sensitive p53 Mutations

### Introduction

P53 is a transcription factor inducible by stress signals such as DNA damage, oncogene activation, and nutrient deprivation. P53 tetramer binds to a specific DNA sequence and activates genes involved in cell cycle, apoptosis, and energy metabolism (Vousden K H, et al. Nat Rev Mol Cell Biol 2007 8:275-83). P53 is the most frequently mutated gene in human cancer (>50% overall mutation rate). Most p53 mutations (˜80%) are amino acid substitutions in the DNA binding domain that cause misfolding or disrupt the DNA binding surface (Leroy B, et al. Hum Mutat 2014 35:672-88; Joerger A C, et al. Annu Rev Biochem 2016 85:375-404). As a result, mutant p53 does not bind DNA or activate target genes.

Mutant p53 is resistant to MDM2-mediated ubiquitination and accumulates to high levels in tumor cells (Peng Y, et al. J Biol Chem 2001 276:40583-90; Li D, et al. Molecular cancer research 2011 9:577-88). Restoring the DNA binding function of mutant p53 is an attractive strategy with significant therapeutic potential (Khoo K H, et al. Nature reviews Drug discovery 2014 13:217-36; Bullock A N, et al. Nat Rev Cancer 2001 1:68-76). However, currently there are no effective mutant p53-targeted drugs approved for clinical use. Wild type (wt) p53 has poor structural stability.

The stability is further reduced in tumor-derived p53 mutants, rendering them unable to bind DNA at 37° C. (Bullock A N, et al. Proc Natl Acad Sci USA 1997 94:14338-42). The development of a yeast assay for detecting p53 mutations unexpectedly revealed that ˜11-15% of tumor-derived p53 mutants are temperature-sensitive (ts) and behaved like wt p53 in the yeast at 32° C. (Shiraishi K, et al. J Biol Chem 2004 279:348-55; Scharer E, et al. Nucleic Acids Res 1992 20:1539-45; Di Como C J, et al. Oncogene 1998 16:2527-39). Most ts p53 mutations are located in the hydrophobic p sandwich core of the DNA binding domain and destabilize the folding at 37° C. (Shiraishi K, et al. J Biol Chem 2004 279:348-55). These mutants are denatured at 37° C. but can regain wt conformation at 32-34° C.

Early work using the mouse p53 ts mutant A135V and the human counterpart A138V in cell culture demonstrated potent apoptosis or cell cycle arrest activities at 32° C. (Yonish-Rouach E, et al. Nature 1991 352:345-7; Pochampally R, et al. J Biol Chem 1999 274:15271-7). Therefore, activating ts p53 mutants may produce strong anti-tumor effects because of their high expression levels. Furthermore, ts p53 mutants are only expressed in tumor cells but not in normal tissues, making them attractive tumor-specific targets for treatment using hypothermia. There are ˜1,700,000 new cancer diagnosis per year in the U.S. If 50% of the new patients have p53 missense mutations and 15% of these mutations are temperature-sensitive, then 7.5% of new cancer patients (127,500 per year) have ts p53 mutations. Therefore, approaches that specifically target tumors expressing ts p53 will have wide impact.

Several mouse models demonstrated that restoring wt p53 expression induced tumor regression. Using p53-ER fusion protein to control p53 activity in Myc-induced lymphomas, Martins and colleagues showed restoring p53 activity for 6 hours led to apoptosis of most tumor cells (Martins C P, et al. Cell 2006 127:1323-34). Continuous p53-ER activation for 7 days extended survival of tumor-bearing mice. Irradiation cooperated with p53 reactivation to increase PUMA expression and further extended survival (Martins C P, et al. Cell 2006 127:1323-34). Using Cre-LoxP to control endogenous p53 expression, Ventura et al showed that restoring wt p53 expression induced rapid apoptosis in lymphomas and senescence in sarcomas, causing tumor regression (Ventura A, et al. Nature 2007 445:661-5). Using inducible shRNA to regulate p53 expression, Xue et al showed that p53 restoration in liver cancer induced tumor regression through senescence and immune clearance (Xue W, et al. Nature 2007 445:656-60). These observations support the hypothesis that restoring ts mutant p53 activity in tumors should also have therapeutic effects.

In principle, cooling tumors to 32° C. will activate ts mutant p53. Due to the locations of most tumors, this requires cooling the body core. Therapeutic hypothermia is the standard of care for resuscitated cardiac arrest patients (Gavrielatos G, et al. Ther Adv Cardiovasc Dis 2010 4:325-33; Dietrich W D, et al. Brain Res 2016 640:94-103; Holzer M. N Engl J Med 2010 363:1256-64). The core temperature of sedated patients is lowered to 32-34° C. using cold blankets for up to 24 hours to improve neurological outcome. Newborn infants with hypoxic-ischemic encephalopathy are cooled for 72 hours for neuro protection (Shankaran S, et al. N Engl J Med 2005 353:1574-84). There are also pre-clinical studies to develop drug-induced hypothermia for treating stroke and brain injury (Zhang M, et al. CNS Neurol Disord Drug Targets 2013 12:371-80). A seminal study showed the A1 adenosine receptor (A1AR) agonist N⁶-cyclohexyladenoxine (CHA) triggers entry into hibernation in arctic ground squirrels (Jinka T R, et al. J Neurosci 2011 31:10752-8). CHA induces transient hypothermia in non-hibernating species such as rats and mice by suppressing thermogenesis and shiver response (Jinka T R, et al. ACS Chem Neurosci 2015 6:899-904; Laughlin B W, et al. Ther Hypothermia Temp Manag 2018 8:108-16). Certain anti-psychotic drugs cause hypothermia as a side effect (Tarahovsky Y S, et al. Psychopharmacology (Berl) 2017 234:173-84; Kreuzer P, et al. J Clin Pharmacol 2012 52:1090-7). The anti-psychotic drug chlorpromazine was used in combination with physical cooling to maintain long duration (2-5 days) hypothermia in patients with brain injury (Jiang J Y, et al. J Cereb Blood Flow Metab 2006 26:771-6). Therefore, hypothermia is an established procedure that can potentially be repurposed for cancer treatment if significant efficacy is demonstrated.

In this report, we tested the therapeutic potential of hypothermia in mice bearing tumor xenografts with ts mutant p53. Using CHA to induce hypothermia for multiple cycles, we observed stasis effects against tumors expressing endogenous ts mutant p53. Furthermore, hypothermia synergized with chemotherapy to induce tumor regression and durable remission in a lymphoma xenograft model. The results suggest hypothermia should be further investigated as a strategy against tumors expressing ts mutant p53.

### Materials and Methods

Cell Lines and plasmids. Cell lines with ts mutant p53 [GA10 (P152L/I232N, ATCC Cat #CRL-2393), SU-DHL-6 (Y234C, ATCC Cat #CRL-2959), NCI-H441 (R158L, ATCC Cat #HTB-174), NCI-H1355 (E285K, ATCC Cat #CRL-5865), NCI-H1963 (H214R/V147D, ATCC Cat #CRL-5982)] were recently purchased from ATCC in 2020 and therefore not tested for mycoplasma or authenticated. Patu8988t (R282W) was provided by Dr. Lixin Wan of Moffitt Cancer Center. All ts p53 mutations in the cell lines were validated by RT-PCR and DNA sequencing. Other cell lines [A549 (wt), MCF7 (wt), U2OS (wt), NCI-H1299 (null), Saos2 (null), MDA-MB-468 (R273H), DU145 (P223L)] were cryopreserved lab stocks last authenticated and tested negative for mycoplasma contamination in 2018. The cells were passaged for <60 days between thawing and experiments. GA-10 and SU-DHL-6 were cultured in RPMI-1640 medium with 20% fetal bovine serum (FBS). All other cell lines were cultured in Dulbecco modified Eagle medium (DMEM) with 10% FBS. H1299 cell lines stably expressing p53 ts or non-ts mutants, were established by infection with pLenti-p53 mutant viruses followed by Zeocin selection (ViraPower T-REX lentiviral expression system, Invitrogen). Each p53 mutation was generated by site-directed mutagenesis on the wt pLenti-p53 plasmid. To knockout p53 in GA10, SU-DHL-6, Patu8988t and NCI-H1963 cells, p53gRNA3 (CACCGCCATTGTTCAATATCGTCCG (SEQ ID NO:1) annealed to AAACCGGACGATATTGAACAATGGC (SEQ ID NO:2)) was cloned into LentiCRISPRv2 vector (Addgene). Cells were infected with lentivirus expressing gRNA, selected with puromycin for single cell clones, and analyzed of p53 expression. Three p53-negative clones were pooled for tumor xenograft analysis.

The profiles of p53 mutations in clinical cases were generated from the Catalogue Of Somatic Mutations In Cancer (COSMIC) database (https://cancer.sanger.ac.uk/cosmic). The tumor cell lines information for p53 ts mutations were acquired from the International Agency for Research on Cancer (IARC) database.

Western blot. Cells were lysed in lysis buffer (50 mM Tris-HCl pH 8.0, 5 mM EDTA, 150 mM NaCl, 0.5% Nonidet P-40, 1×protease inhibitor cocktail) and centrifuged for 10 minutes at 14,000×g, and the insoluble debris was discarded. Cell lysate (10-50 pg of protein) was fractionated by SDS-PAGE and transferred to Immobilion P filters (Millipore). The filter was blocked for 1 hr with phosphate buffered saline (PBS) containing 5% non-fat dry milk and 0.1% Tween 20, incubated with primary and secondary antibodies, and the filter was developed using SuperSignal reagent (Thermo Scientific). MDM2 was detected using monoclonal antibody 3G9 produced in house. Other markers were detected using commercial antibodies: Actin (Sigma A5441), p53-DO1 (BD Pharmingen #554293), p21 (BD Pharmingen #556430), PUMA (CellSignaling #12450), PARP (BD Pharmingen #556362), p53 pSer15 (CellSignaling #9284), p53 acetyl-Lys382 (CellSignaling #2525).

RNA isolation and quantitative RT-PCR. Total RNA was extracted using the RNeasy Mini kit (Qiagen). cDNAs were prepared by reverse transcription of total RNA using Applied Biosystems™ High-Capacity cDNA Reverse Transcription Kit. The products were used for SYBR Green real-time PCR using the following primers: GAPDH (TCACCACCATGGAGAAGGC (SEQ ID NO:3) and GCTAAGCAGTTGGTGGTGCA (SEQ ID NO:4)), p14-ARF (TCTTGGTGACCCTCCGGATTCGG (SEQ ID NO:5) and TCAGCCAGGTCCACGGGCAGA (SEQ ID NO:6)).

Luciferase reporter assay. H1299 cells (50,000/well) were seeded in 24-well plate and transfected with 2 ng pLenti-p53 plasmid (wt or mutant), 5 ng CMV-lacZ plasmid, and 20 ng p53-responsive luciferase reporter plasmid driven by MDM2, p21, or PUMA promoters. Transfection was achieved using Lipofectamine 3000 (Invitrogen). Twenty-four hours after transfection at 37° C., cells were transferred to 32° C. for 20 hrs, and analyzed for luciferase and beta-galactosidase activity. The transcriptional activity of p53 was indicated by the ratio of luciferase/beta-galactosidase activity.

Chromatin immunoprecipitation (ChIP). ChIP assay was performed using standard procedure. Crosslinked p53-DNA complexes were immunoprecipitated with DO-1 antibody. Samples were subjected to SYBR Green real-time PCR analysis using forward and reverse primers for the p53 binding sites in the p21 promoter (AGGAAGGGGATGGTAGGAGA (SEQ ID NO:7) and ACACAAGCACACATGCATCA (SEQ ID NO:8)), and PUMA promoter (CTGTGGCCTTGTGTCTGTGAGTAC (SEQ ID NO:9) and CCTAGCCCAAGGCAAGGAGGAC (SEQ ID NO:10)).

Cell proliferation and viability assays. DNA replication was analyzed using ³H-thymidine incorporation assay. Cells were plated in 12-well plate (5×10⁵/well) and incubated at 37° C. or 32° C. for 48 hrs. ³H-thymidine (4 μCi/well, 25 Ci/mmol, PerkinElmer) was added to culture media and incubated at 37° C. for 3 hrs. Cells were washed twice with cold PBS and treated with 1 ml of ice-cold 5% Trichloroacetic acid at 4° C. for 30 min. After washing twice with PBS, the cells were lysed in 1 ml of 1 N NaOH at 37° C. for 30 min. The incorporated ³H-thymidine was analyzed by liquid scintillation counting. MTS cell viability analysis was performed using Celltiter 96 AQueous One Solution reagent following manufacturer instructions (Fisher #PR-G3580).

In vitro p53 refolding and DNA binding assay. XL1-Blue cotransformed with pBB540 (Addgene #27393, expressing GrpE, ClpB) and pBB550 (Addgene #27396, expressing DnaK, DnaJ, GroESL) was cultured in LB medium containing chloramphenicol (30 pg/ml) and spectinomycin (50 pg/ml) to OD600=0.6 at 37° C. and induced with 0.1 mM IPTG at 18° C. for 18 hrs. Cell pellet from 250 ml culture was suspended in 5 ml phosphate buffer (100 mM potassium phosphate pH7.8, 0.01% Triton X-100, 1 μM ZnCl₂. 1 mM DTT), disrupted by sonication, and centrifuged at 14,000×g for 10 min at 4° C. to prepare supernatant containing chaperones (stored at −80° C. with 5% glycerol). The p53 in vitro DNA binding assay contained ZF-Nluc (luciferase aa 1-437 fused to C-terminus of a zinc finger protein and cloned into pET28 vector), p53-Cluc (luciferase aa 398-550 fused to C-terminus of p53 or R282W and R175H mutants and cloned into pET28), ZPBS12 plasmid DNA [pUC57 vector with a 560 bp DNA insert containing 12 copies of ZF binding site (ATGTAGGGAAAAGCCCGG (SEQ ID NO:11)) and p53 binding site (GAACATGTCCCAACATGTTG (SEQ ID NO:12)) with various spacing (0, 2, 4, 6, 8, 10 bp)]. BL21DE3 transformed with p53-Cluc or ZF-Nluc were cultured to OD600=0.6 at 37° C. in LB medium containing 150 μM ZnCl₂, and induced with 0.1 mM IPTG for 20 hrs at 16° C. Pelleted cells were sonicated in lysis buffer [50 mM HEPES (pH 7.5), 150 mM NaCl, 0.1% Nonidet P-40, 5% glycerol, 10 μM ZnCl₂, 1 mM DTT] and centrifuged at 14,000×g for 10 min at 4° C. The lysate was diluted to ˜10 ng/μl total protein with dilution buffer [4.25% (vol/vol) 0.2 M NaH₂PO₄, 45.75% (vol/vol) 0.2 M Na₂HPO₄, 5% glycerol, 1 μM ZnCl₂, 1 mM DTT]. The diluted BL21DE3 extract (10 μl, ˜200 ng protein) containing p53-Cluc or R282W-Cluc was incubated at 34° C. for 20 min to inactivate the ts p53. The heat-treated R282W-Cluc was mixed with 10 μl XL1-Blue extract containing chaperones (or XL1-Blue control extract), 5 mM ATP, and incubated at 23° C. for 2 hrs for refolding. The refolded p53-Cluc mixture was combined with 200 ng ZF1-Nluc extract and 50 ng ZPBS12 plasmid DNA in a 40 μl DNA binding reaction mixture and incubated for 30 min at 23° C. Luciferase substrate A (25 mM glycylglycin pH7.8, 15 mM potassium phosphate pH7.8, 15 mM MgSO₄, 4 mM EGTA, 2 mM ATP, 1 mM DTT) was combined with equal volume of luciferase substrate B (0.4 mg/ml D-luciferin, 25 mM glycylglycin pH7.8, 2 mM DTT), and 30 μl substrate mixture was added to the DNA binding reaction mixture. After incubating for 10 min at 23° C., luciferase signal was measured using a microplate luminometer.

Animal experiment. Animal experiments were reviewed and approved by the University of South Florida IACUC. Athymic female nude mice (6-week old, Athymic Nude-Foxn1nu, Envigo) were injected subcutaneously with 0.1 ml 1:1 slurry of Matrigel (VWR 47743-715) and 1×10⁷ cells in PBS at each site. The inoculated mice were housed in a cabinet set to 28° C. (ARIA BIO-C36 ventilated cabinet, TECNIPLAST) to ensure that tumors develop at ˜37° C. Each mouse received injections at 2 sites, tumors formed at >80% of sites injected with GA10 cells. N⁶-cyclohexyladenosine (CHA, Sigma C9901) was dissolved at 20 mg/ml in 25% (2-Hydroxypropyl)-β-cyclodextrin (w/v, Sigma H107) and heated at 65° C. for 10 min to ensure dissolution. Camptothecin (CPT, Santa Cruz Biotechnology sc-200871B) was freshly dissolved in 1 N NaOH, diluted to ˜0.5 mg/ml with saline, and adjusted to pH10 using HCl. To induce hypothermia, 0.2 ml saline+10% glucose containing CHA and CPT was administered by intra peritoneal injection to provide a CHA dosage of 10 mg/kg body weight and CPT (when indicated) dosage of 1.5 mg/kg body weight. The mice were kept in the 28° C. cabinet and monitored using infrared thermal imaging camera (FLIR ONE Pro, FLIR® Systems, Inc.) to verify that body temperature was maintained at ˜32° C. A second injection of CHA (10 mg/kg) in 0.4 ml saline+10% glucose was given after 10 hours to maintain hypothermia for ˜24 hours. A third injection of CHA (10 mg/kg) in 0.4 ml saline+10% glucose was given after 10 hours to maintain hypothermia for ˜32 hours. After regaining normal body temperature, the mice were kept for 2-4 days in the 28° C. cabinet to allow weight recovery (˜10% weight loss occurred during hypothermia due to loss of mobility and food intake). The treatment was repeated 4-5 times (24 hr×6 or 32 hr×5 format). In each subsequent round CHA dosage was increased by 25% to compensate for the gradual loss of sensitivity.

Flow cytometry. GA-10 control or p53-knockout cells were cultured at 37° C. or 32° C. respectively for 48 hrs. The cells were collected by centrifuging at 3000×g for 5 min, washed with PBS, stained with Annexin V-PE and 7-AAD, and subjected to FACS analysis using FACSCalibur flow cytometer (BD BioSciences). The data were analyzed using FlowJo software (TreeStar).

Immunohistochemistry. Mice bearing GA-10 xenograft tumors were treated with hypothermia (with or without CPT) for 24 hrs and euthanized for tumor collection. The tumor samples were fixed in formalin and paraffin sections were prepared. After de-paraffinization and rehydration with xylene and a graded ethanol series, tissue sections were analyzed with TUNEL staining using the In situ Apoptosis Detection Kit (Abcam ab206386).

Statistical analysis. The experimental results were presented as mean±standard deviation (SD), and Student's t-test was used to evaluate differences between groups. P<0.05 was considered statistically significant.

### Results

Somatic p53 ts mutation frequency in cancer. P53 A135V (mouse) and A138V (human) mutants were frequently used for controlling p53 activity in culture (Yonish-Rouach E, et al. Nature 1991 352:345-7; Yamato K, et al. Oncogene 1995 11:1-6). The human mutant V143A is also a ts mutation (Friedlander P, et al. J Biol Chem 1996 271:25468-78). A138V and V143A mutations occur at relatively low frequency in cancer, thus have limited clinical significance. We recently observed that R282W also behaved as a ts mutant similar to A138V in inducing MDM2 and p21 at 3200 (FIG. 8A-8D). As one of six p53 hotspot mutants, R282W alone accounts for 3.3% (820/24,679) of p53 missense mutations in cancer according to the COSMIC database (FIG. 1A, Table 1). Therefore, we analyzed the p53 mutations curated by the COSMIC database to obtain an estimate of ts mutation frequency in cancer.

TABLE 1
Frequency of ts p53 mutations in cancer.

| Ts | Cases in | % of | Ts | Cases in | % of |
|---|---|---|---|---|---|
| mutation | COSMIC | ^{a}24,679 | mutation | COSMIC | ^{a}24,679 |
| S99FP | 4 | 0.02% | S215GIN | 91 | 0.37% |
| Y126C | 27 | 0.11% | V216L | 18 | 0.07% |
| N131HS | 8 | 0.03% | V217G | 6 | 0.02% |
| C135W | 36 | 0.15% | P219LS | 9 | 0.04% |
| A137V | 1 | 0.00% | P223AHLRT | 14 | 0.06% |
| A138VT | 39 | 0.16% | C229G | 1 | 0.00% |
| V143A | 19 | 0.08% | T231ANPS | 2 | 0.01% |
| W146G | 3 | 0.01% | I232FM | 13 | 0.05% |
| P151AS | 136 | 0.55% | Y234CH | 134 | 0.54% |
| P152LT | 109 | 0.44% | S240R | 7 | 0.03% |
| G154V | 65 | 0.26% | M246V | 67 | 0.27% |
| T155IN | 54 | 0.22% | N2471Y | 13 | 0.05% |
| R156GP | 37 | 0.15% | R249K | 20 | 0.08% |
| R158CGHL | 283 | 1.15% | P250L | 70 | 0.28% |
| M160IKRT | 9 | 0.04% | I251LT | 4 | 0.02% |
| A161GSV | 23 | 0.09% | L252FH | 11 | 0.04% |
| Q165LPR | 5 | 0.02% | T253AINS | 11 | 0.04% |
| V172FAGL | 46 | 0.19% | I254FMT | 15 | 0.06% |
| R175GL | 61 | 0.25% | I255FM | 38 | 0.15% |
| A189PS | 3 | 0.01% | T256AIR | 4 | 0.02% |
| P190T | 11 | 0.04% | L257V | 9 | 0.04% |
| H193RQY | 231 | 0.94% | N268IT | 2 | 0.01% |
| R196L | 3 | 0.01% | F270IV | 27 | 0.11% |
| V197LM | 16 | 0.06% | E271AGV | 12 | 0.05% |
| Y205NH | 18 | 0.07% | V272MAL | 203 | 0.82% |
| D208AV | 22 | 0.09% | V274A | 38 | 0.15% |
| T211AP | 7 | 0.03% | R282W | 820 | 3.32% |
| F212V | 2 | 0.01% | R283HP | 46 | 0.19% |
| R213GP | 14 | 0.06% | E285KDGV | 244 | 0.99% |
| H214RDLNY | 118 | 0.48% | E286GKQV | 206 | 0.83% |
| S215GIN | 91 | 0.37% | L289P | 2 | 0.01% |
| Total | 1501 | 6.08% | Total | 2157 | 8.74% |

^{a}Total number of tumors with missense p53 mutation in COSMIC.

A previous study by Shiraishi et al using a yeast transactivation assay at 30° C. identified 142 p53 ts mutations out of a library of 2,314 mutants covering all single nucleotide mutations in the DNA binding domain (Shiraishi K, et al. J Biol Chem 2004 279:348-55). The 142 ts mutants were observed in 10.4% of 12,032 tumors with mutant p53 (Shiraishi K, et al. J Biol Chem 2004 279:348-55). However, the yeast assay did not identify several ts mutants validated in human cells such as R282W, A138V, and V143A. Inclusion of these ts mutants into the yeast-generated ts mutant list and analysis of the current COSMIC database showed that ˜14.8% of tumors with p53 missense mutations express ts p53 (3,658/24,679, Table 1, Table 2). Lung and skin tumors had higher than average ts mutation frequency of 18.4% and 18.9% respectively (Table 2). The 3,658 ts mutations affected 62 amino acid residues in the 300-residue DNA binding domain (Table 1). The majority of ts mutations (68%, 2,484/3,658) occurred at 10 ts hotspot codons (FIG. 1B).

TABLE 2
Ts p53 mutation frequency in the COSMIC database.

| | All tumors | Lung | Skin | Brain | Ovary | Breast |
|---|---|---|---|---|---|---|
| Missense mutations | 24679 | 2715 | 709 | 1295 | 1278 | 2191 |
| Ts mutations | 3658 | 500 | 134 | 208 | 152 | 268 |
| % ts | 14.8% | 18.4% | 18.9% | 16.1% | 11.9% | 12.2% |

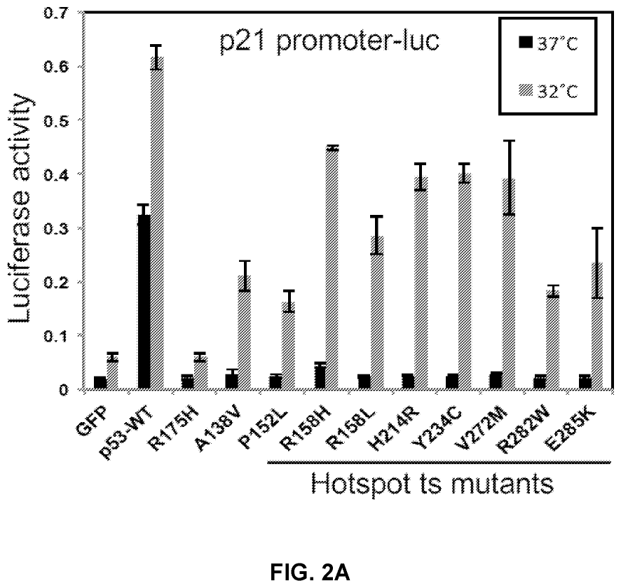

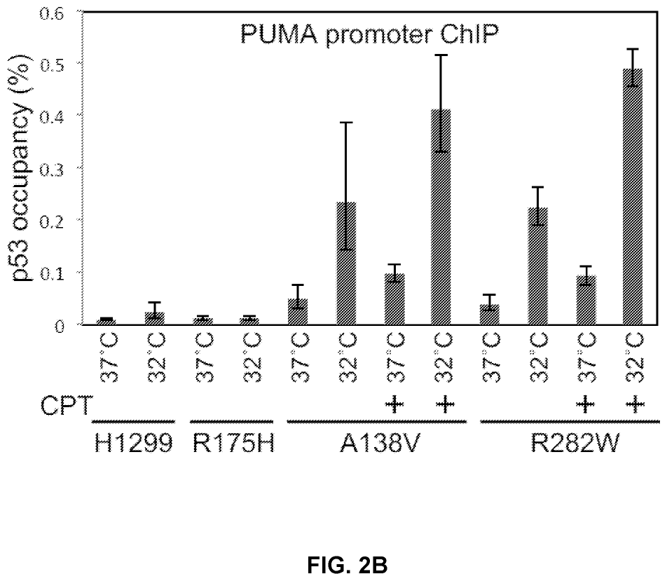

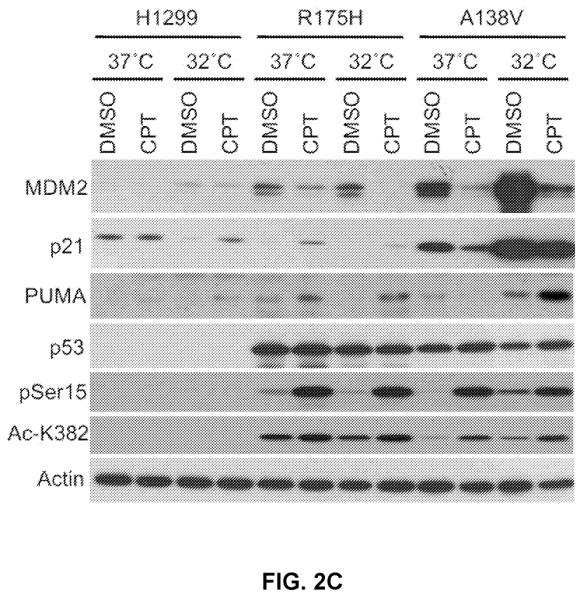

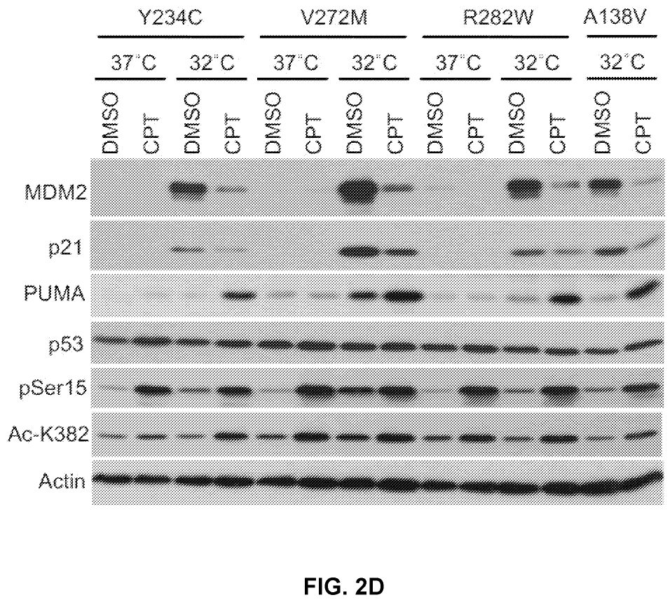

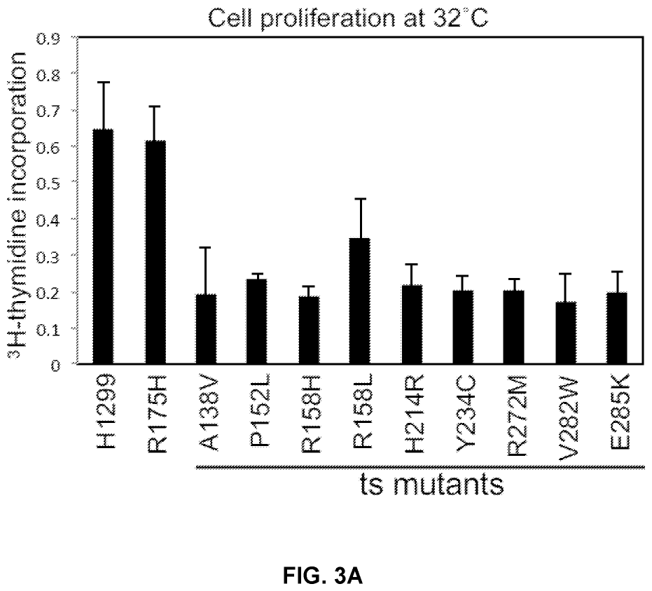

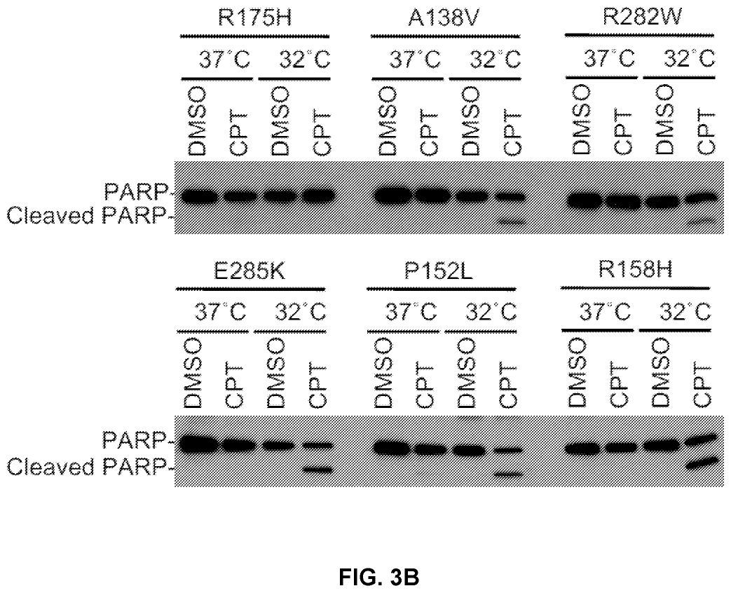

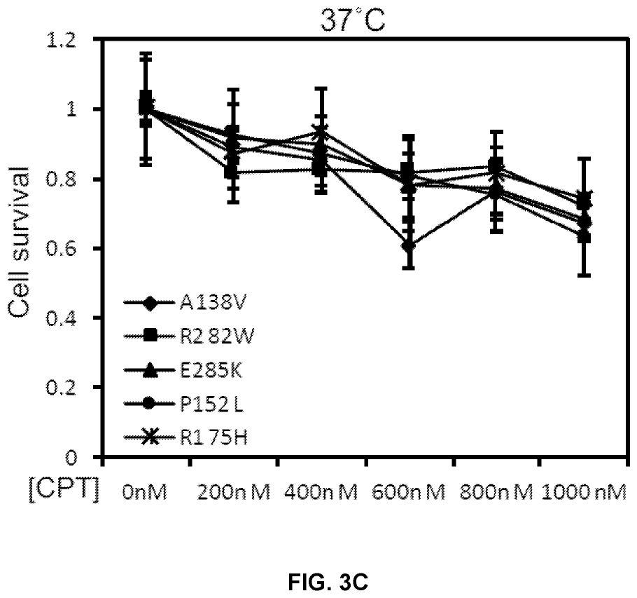

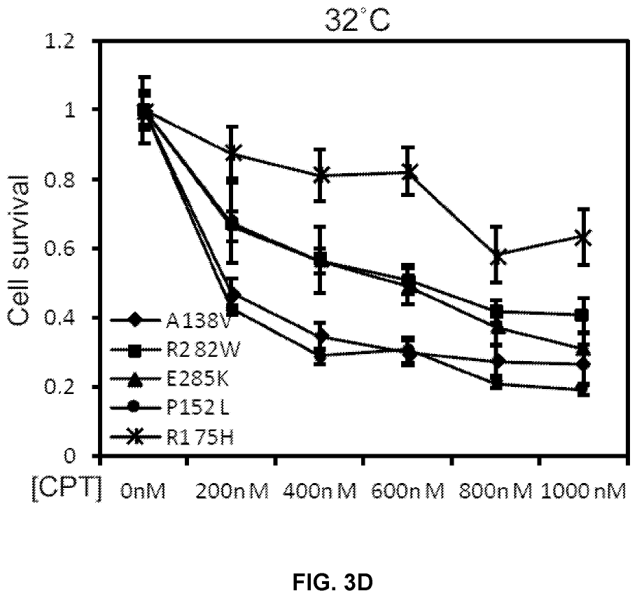

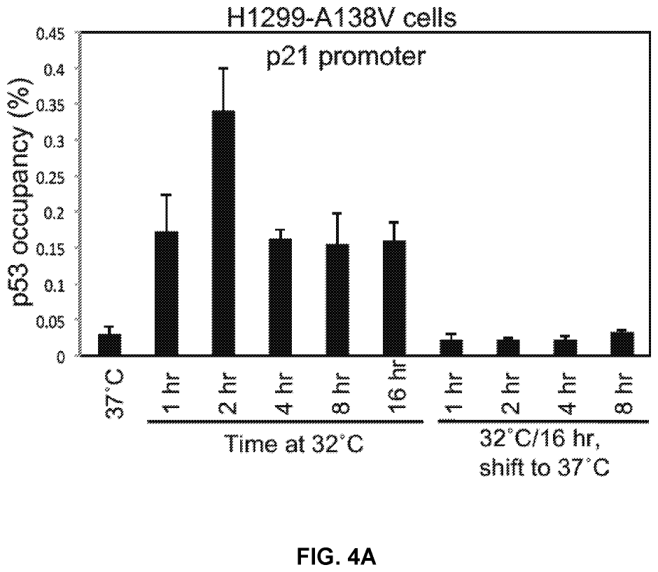

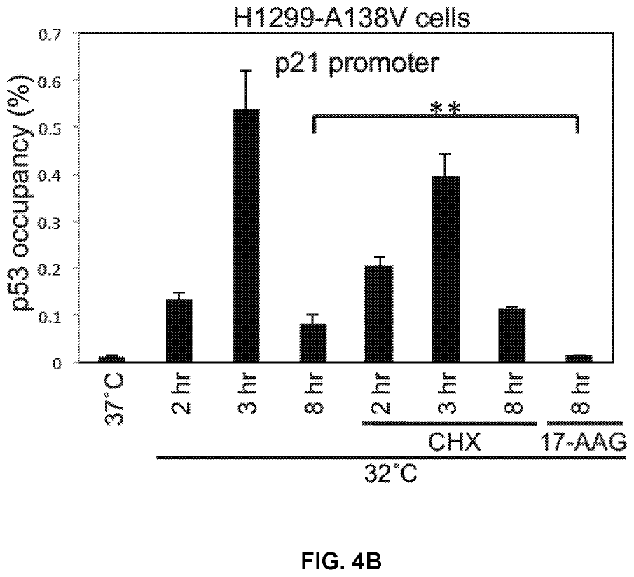

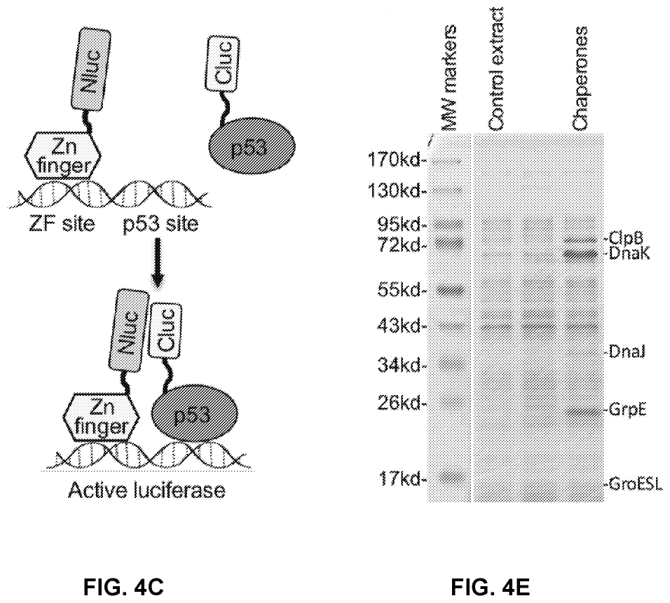

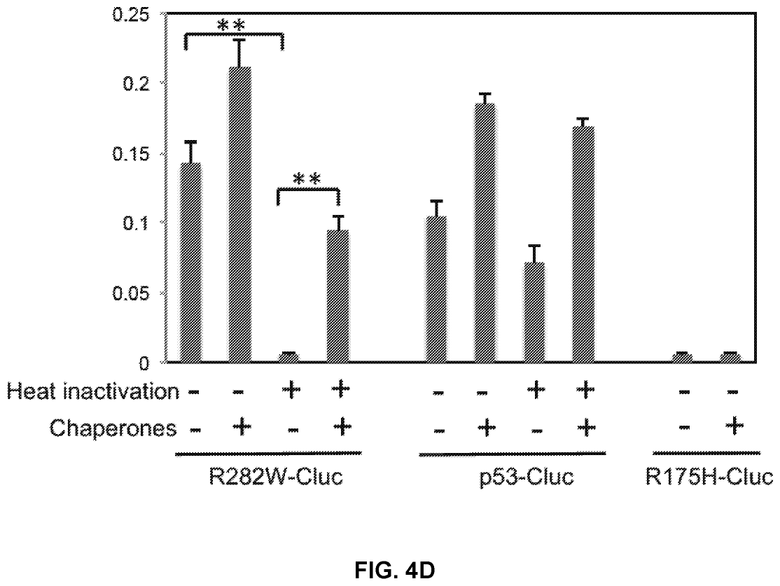

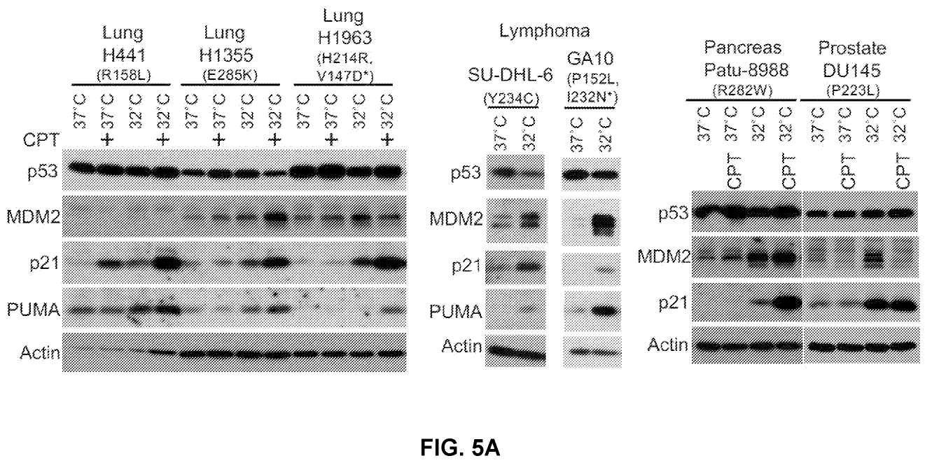

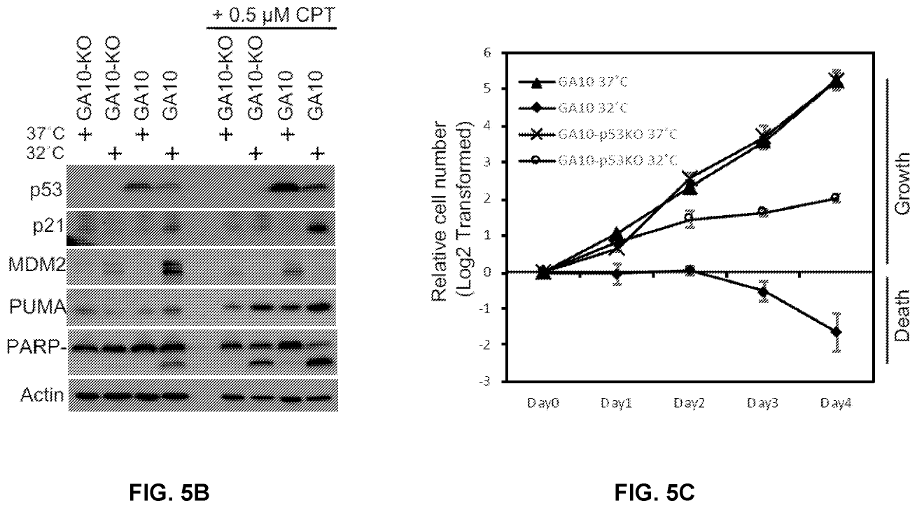

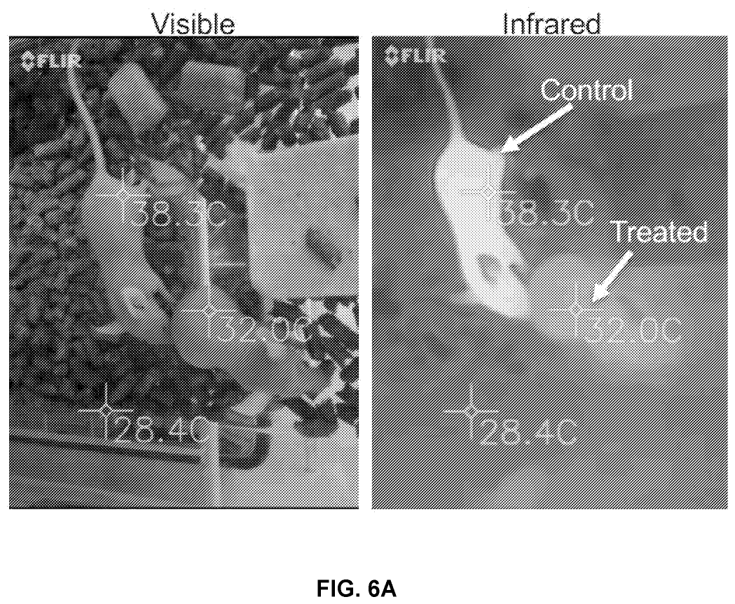

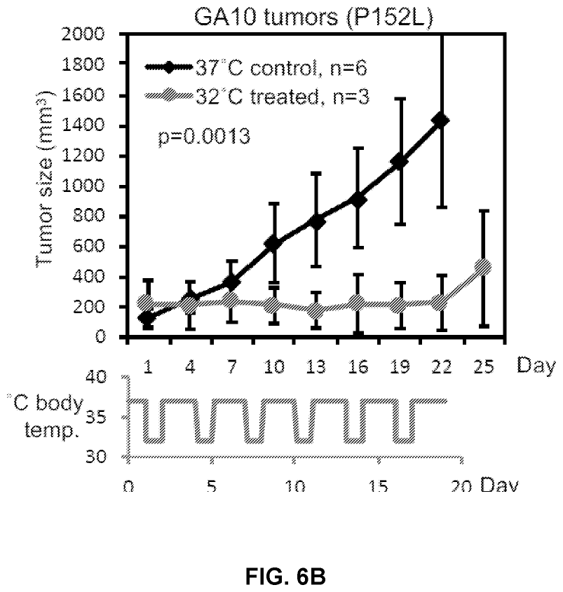

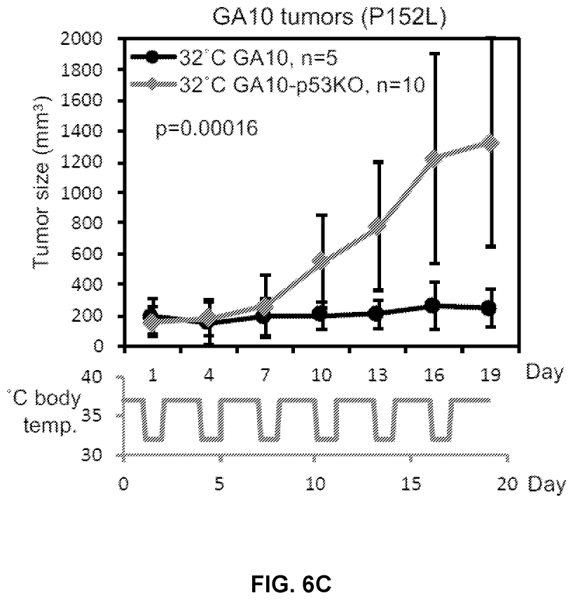

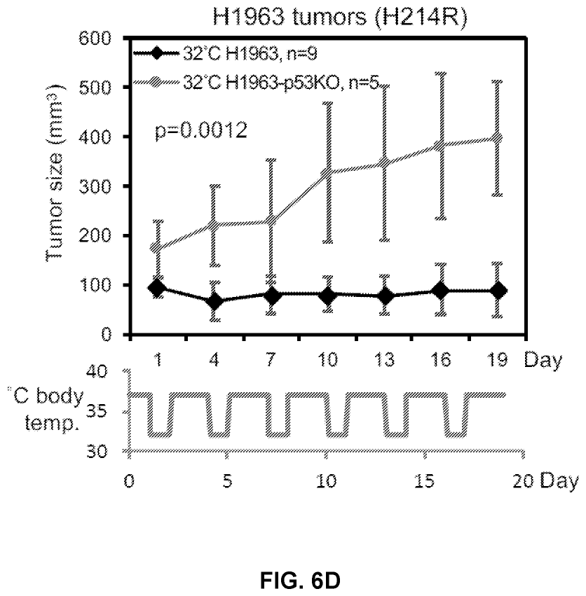

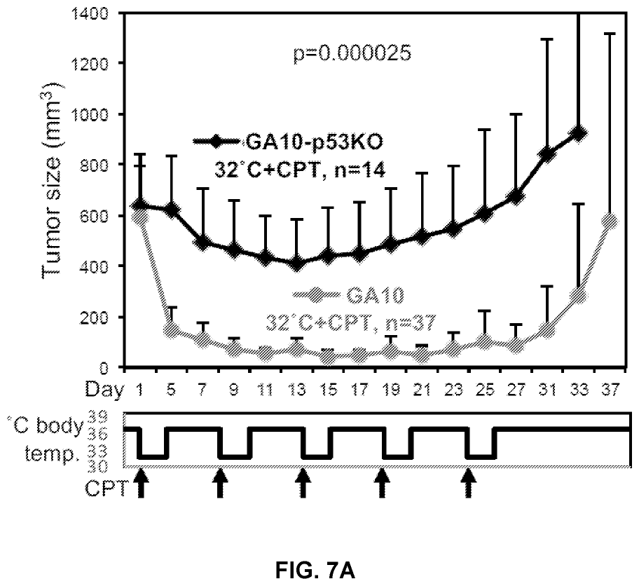

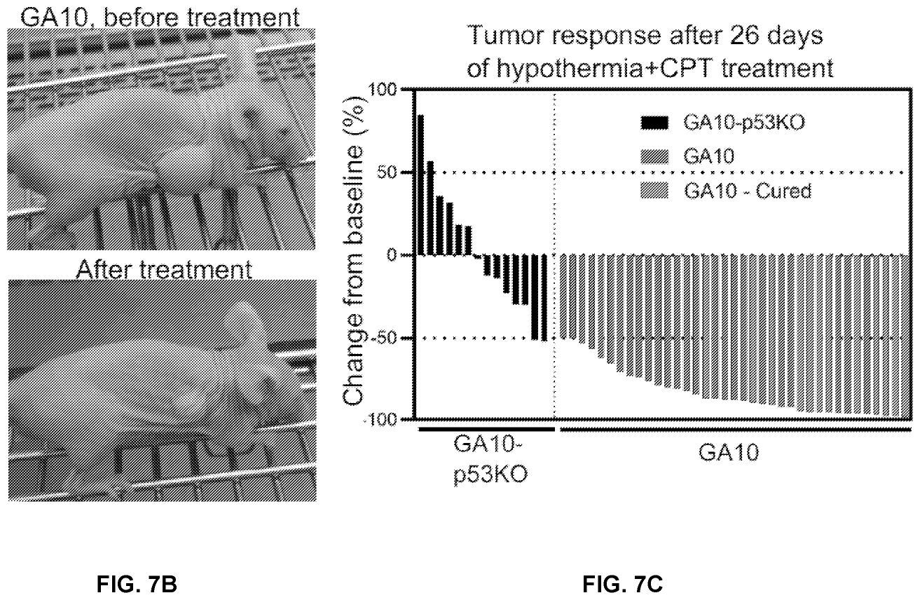

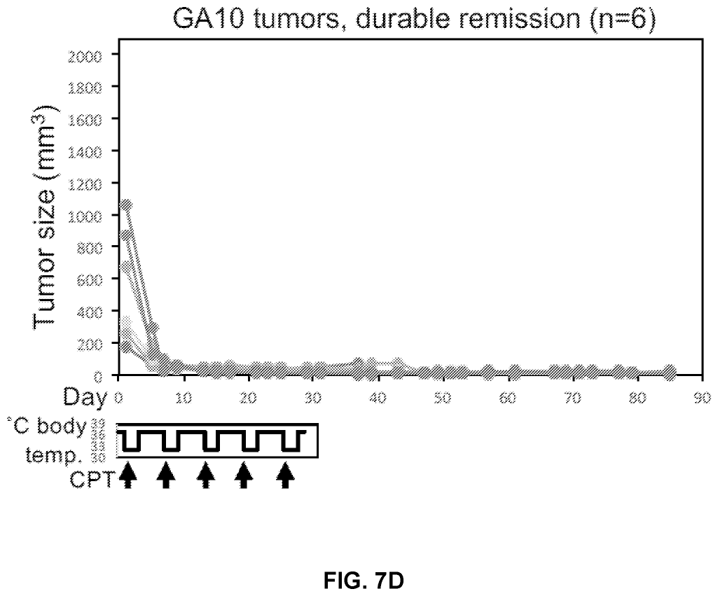

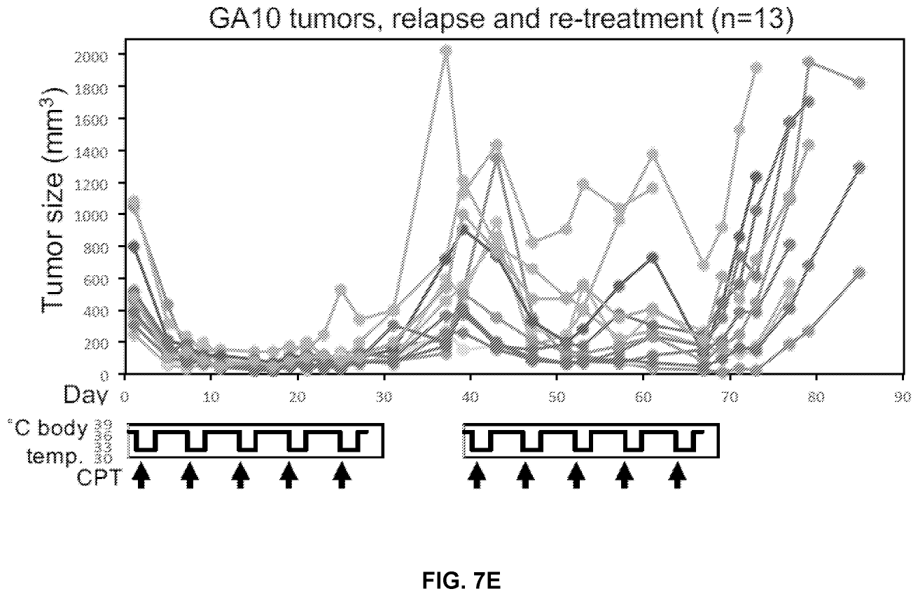

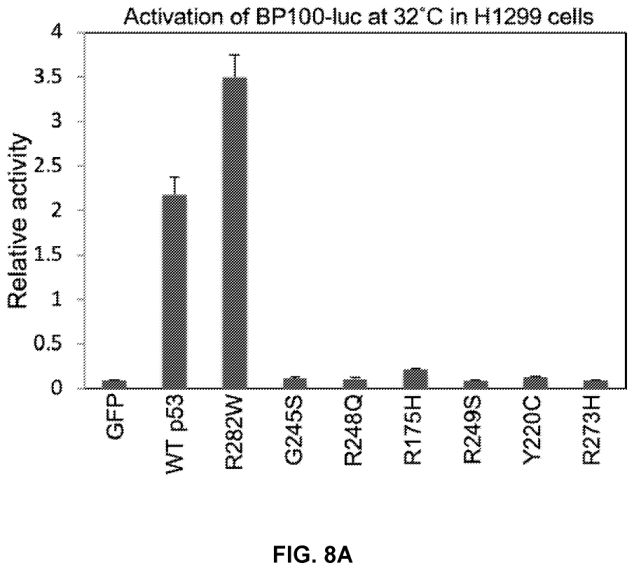

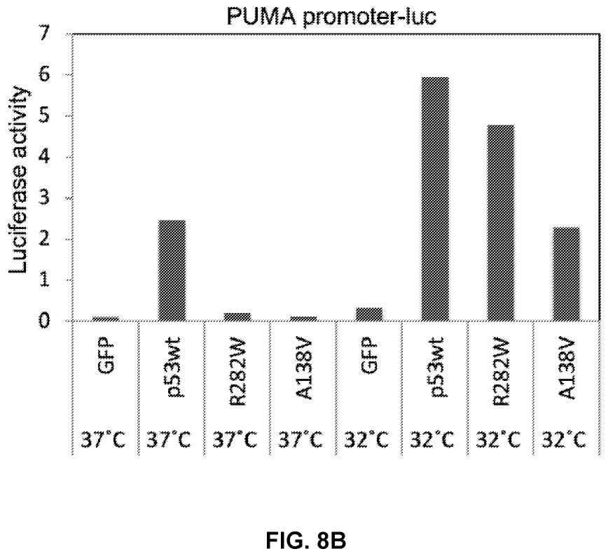

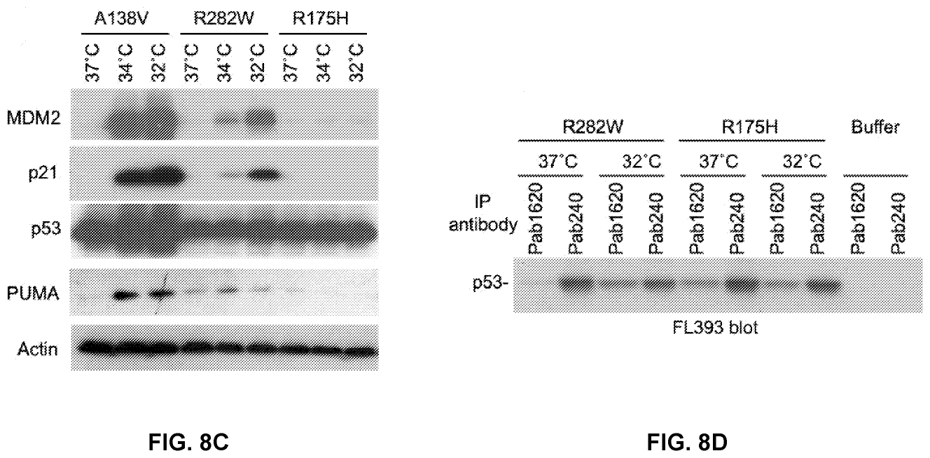

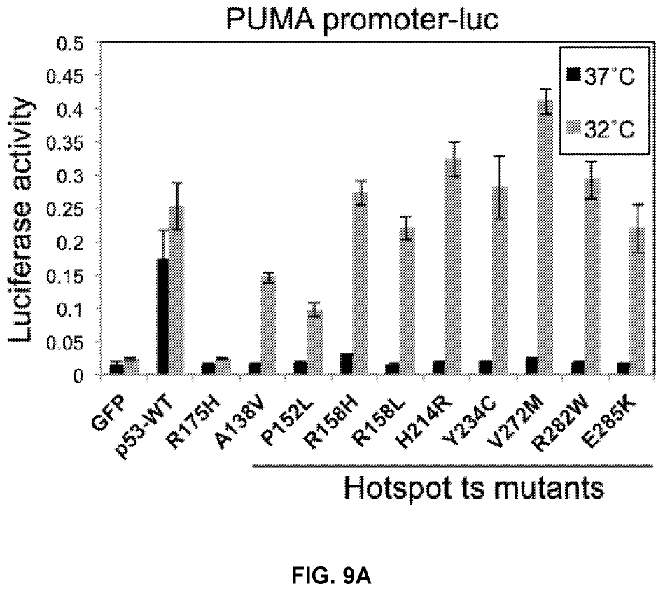

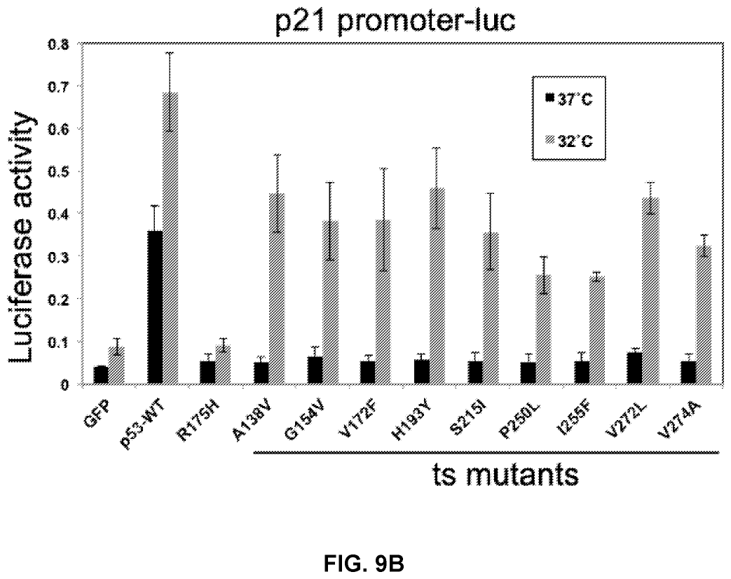

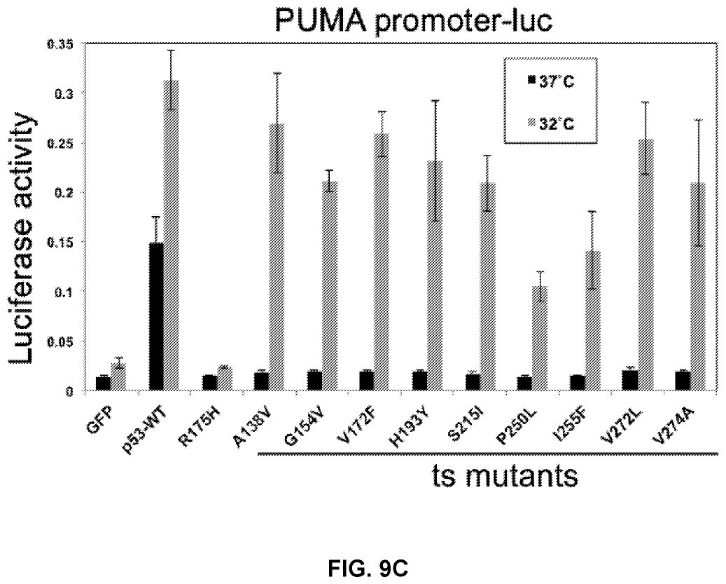

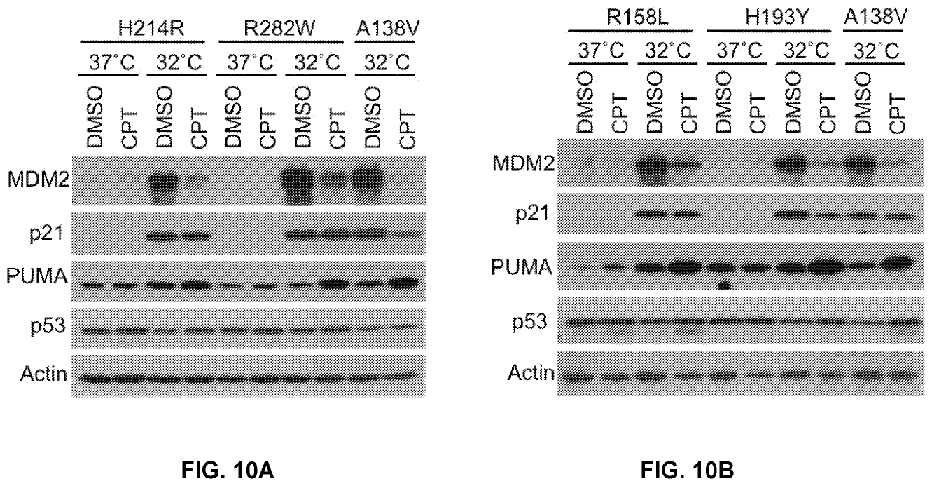

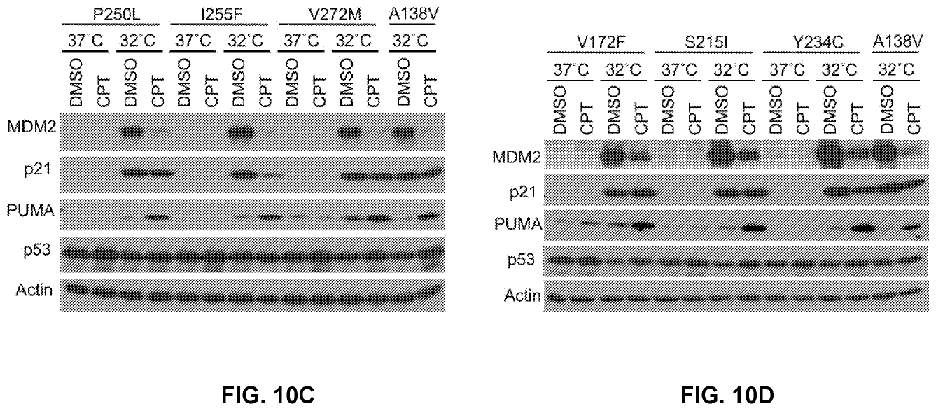

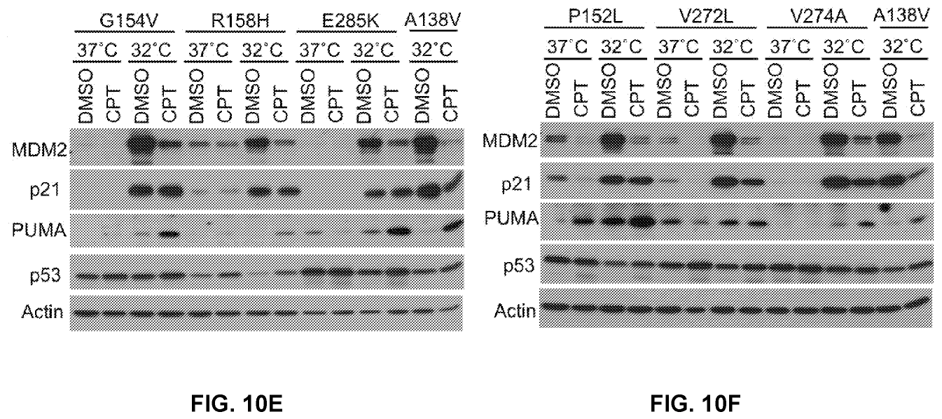

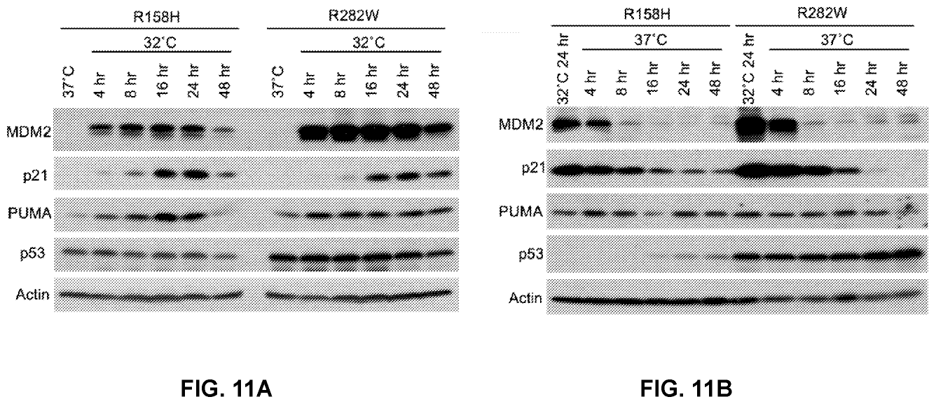

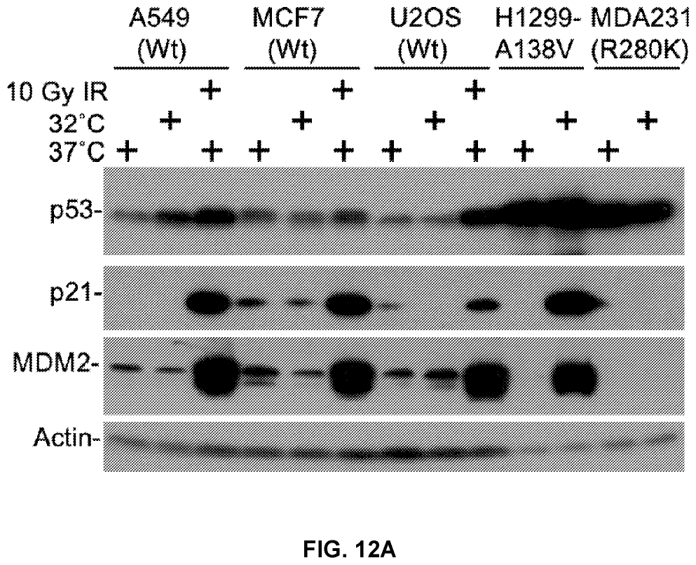

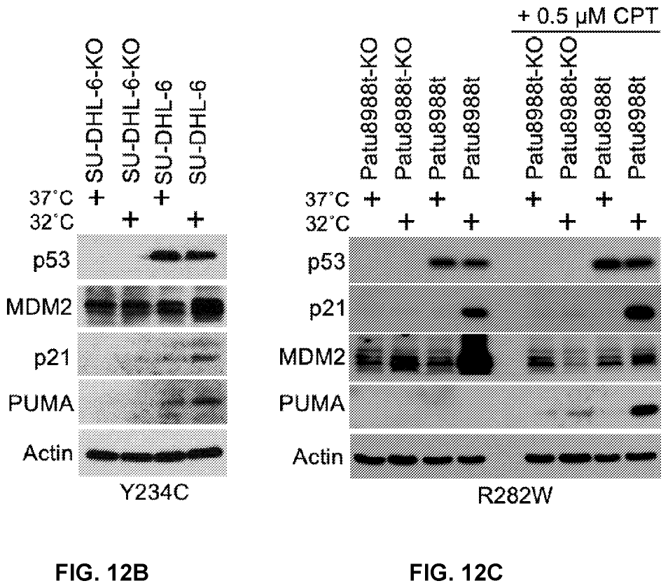

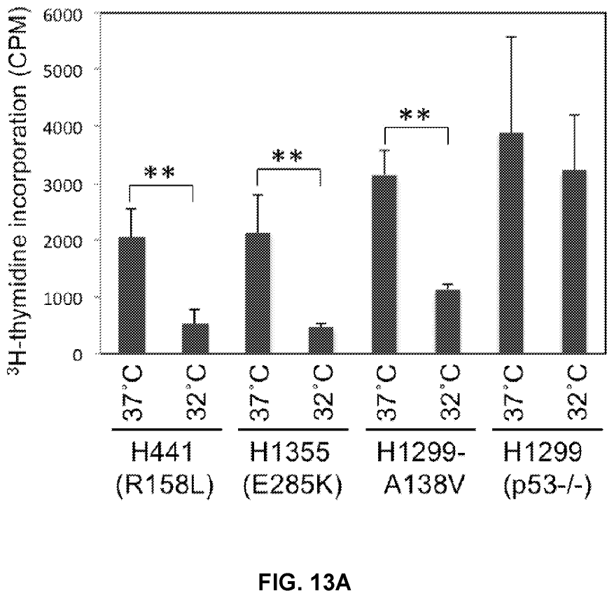

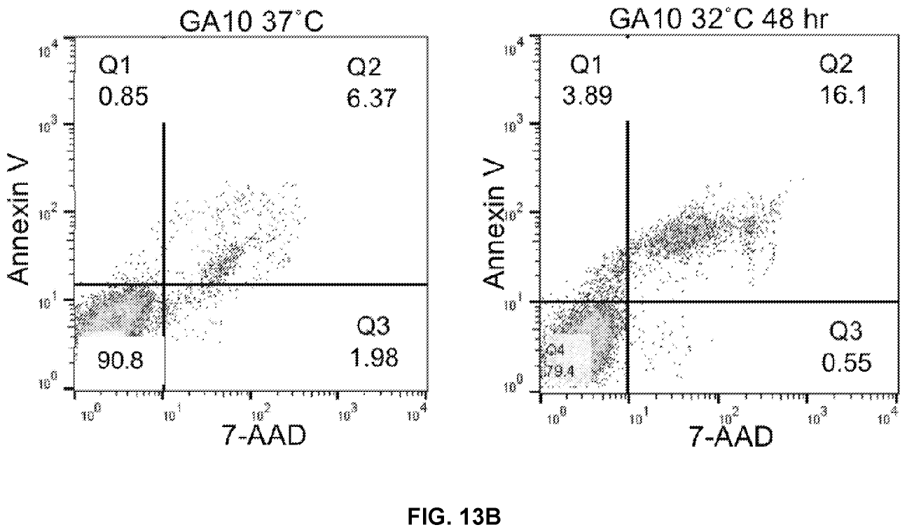

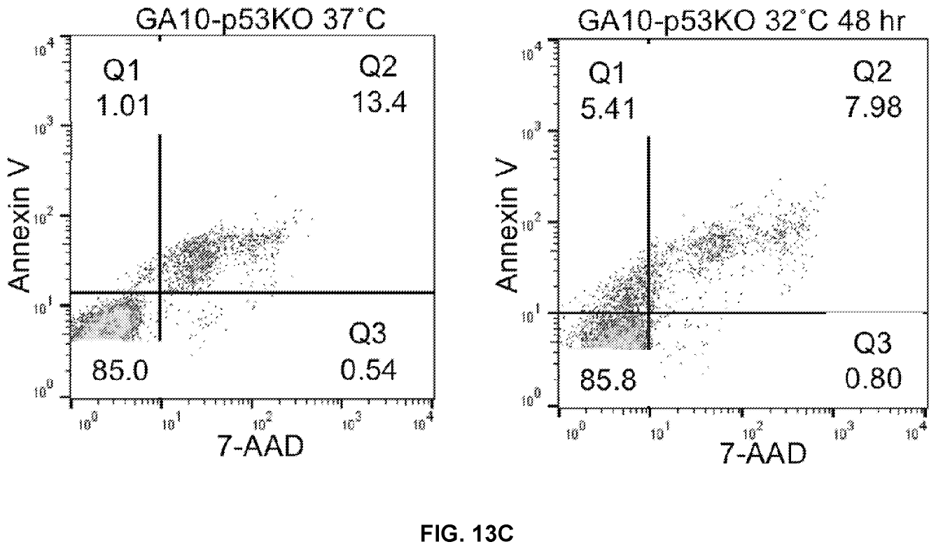

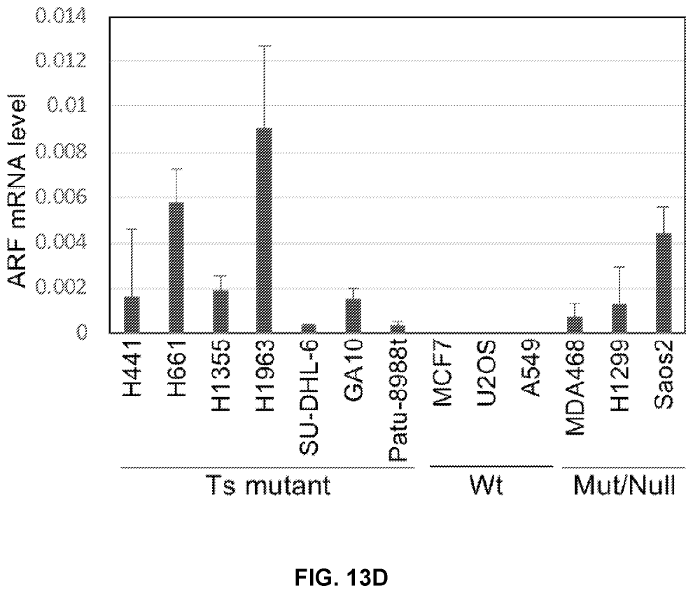

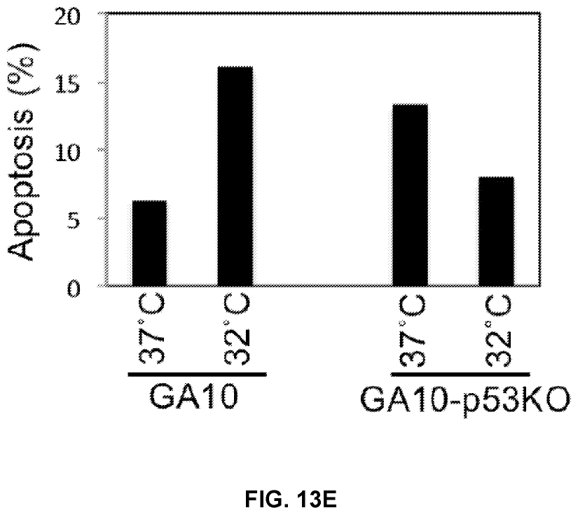

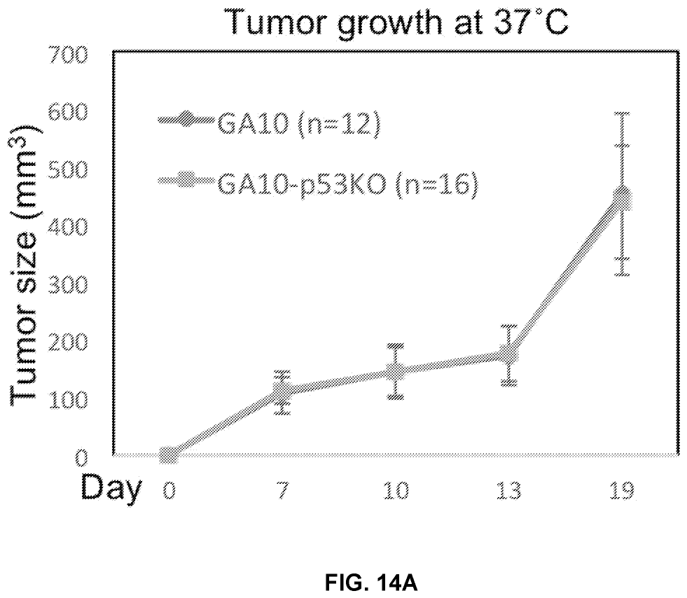

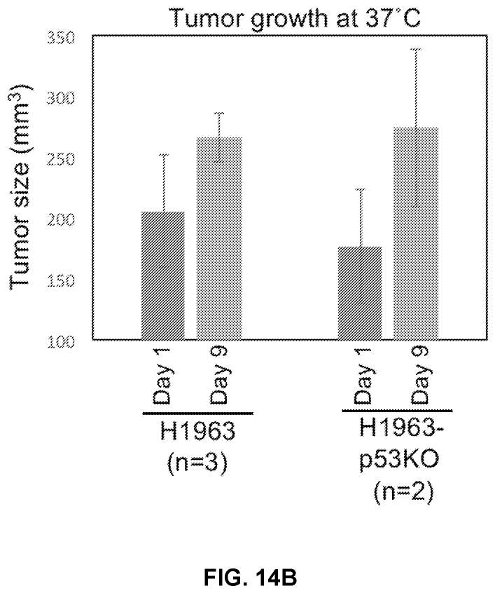

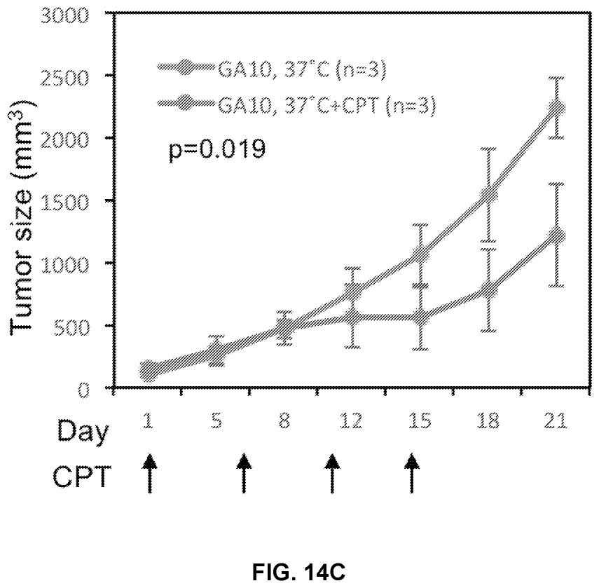

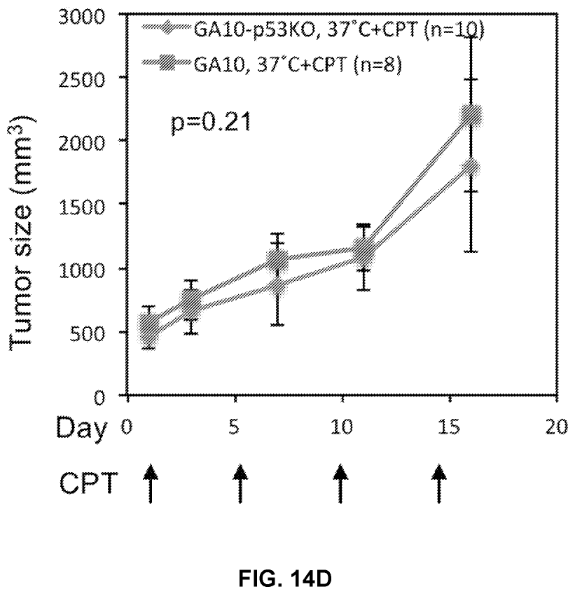

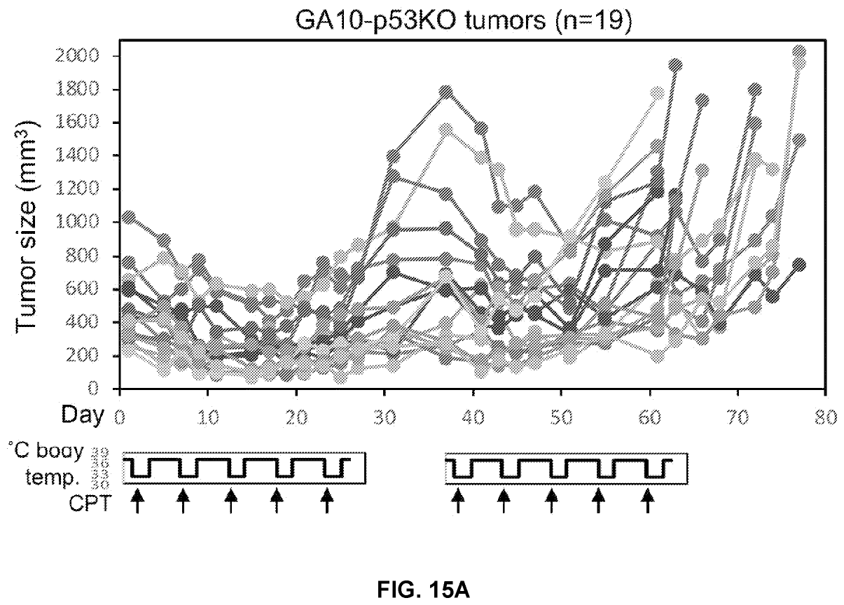

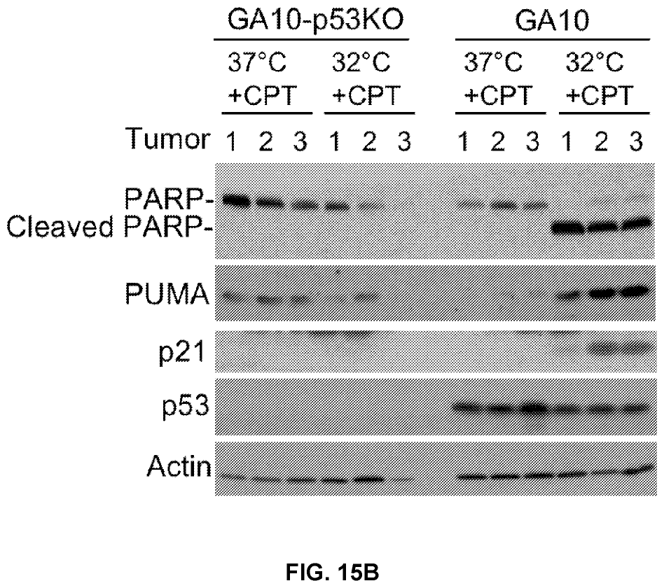

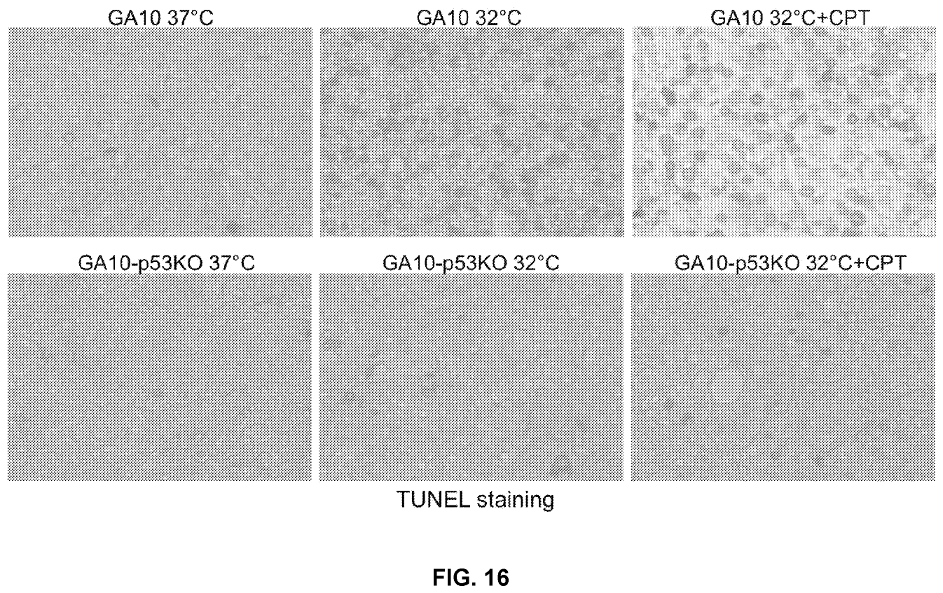

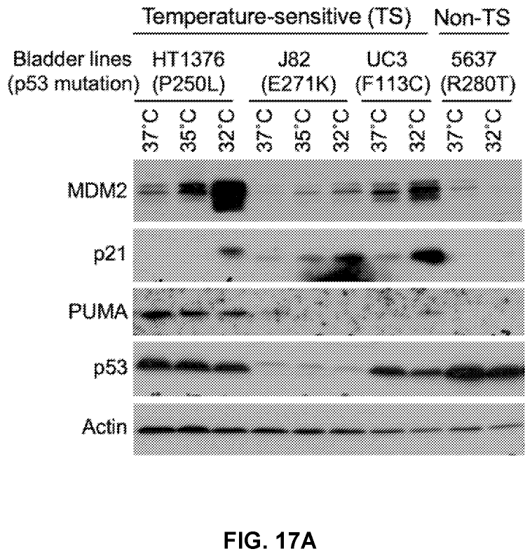

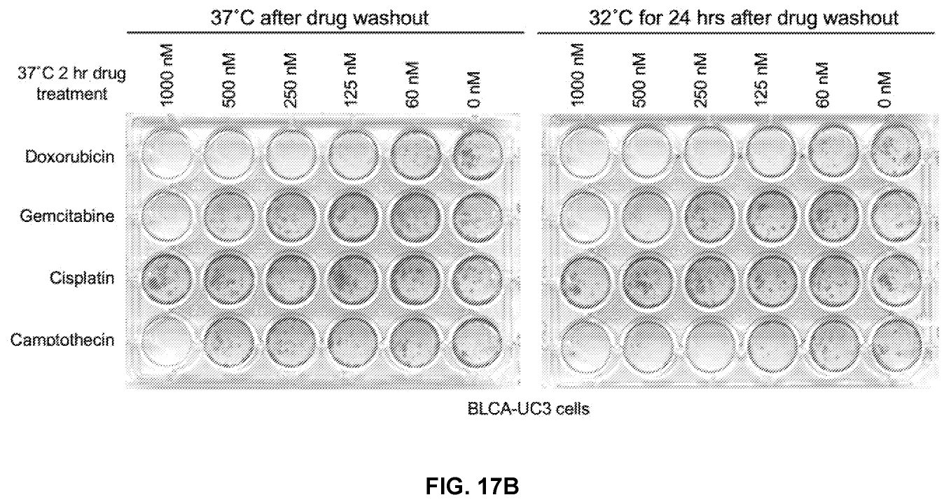

Validation of ts p53 mutants from cancer. We selected 17 most frequently observed ts mutants for validation in p53-null H1299 cells. These ts mutants together represent 9.6% (2,358/24,679) of tumors with p53 missense mutations (Table 3), or 64% (2,358/3,658) of tumors with ts p53 mutations. The ability of these mutants to activate the p21 and PUMA promoters at 32° C. were confirmed in reporter assays after transient expression (FIG. 2A, FIG. 8A-8C). The ts mutants were also stably expressed in H1299 at 37° C. and tested for induction of MDM2, p21 and PUMA after shifting to 32° C. Although all ts mutants activated the PUMA promoter-luc reporter in transient transfection assay, induction of endogenous PUMA in stably transfected H1299 was modest and in most cases could be stimulated by DNA-damaging agent camptothecin (CPT. FIG. 2B, 2C, FIG. 9A-9F). CPT appeared to synergize with ts p53 through several mechanisms. ChIP analysis showed that while 32° C. alone modestly activated PUMA promoter binding by the ts mutants, CPT treatment further stimulated the binding (FIG. 2D). In the absence of p53, CPT modestly stimulated PUMA expression (FIG. 2B), suggesting it also primed the PUMA promoter for activation by p53. Furthermore, CPT stimulated ts p53 Ser15 phosphorylation and K382 acetylation, which may increase its DNA binding and transcriptional activity (FIGS. 2B,2C).

TABLE 3
Seventeen most frequent p53 ts mutants.

| Validated | Cases in | | ^{b}Tumor |
|---|---|---|---|
| ts mutants | COSMIC | ^{a}Frequency | cell lines |
| R282W | 820 | 3.32% | 32 |
| E285K | 252 | 1.02% | 13 |
| V272M | 146 | 0.59% | 10 |
| Y234C | 144 | 0.58% | 9 |
| R158H | 129 | 0.52% | 9 |
| R158L | 127 | 0.51% | 8 |
| P152L | 124 | 0.50% | 4 |
| H214R | 122 | 0.49% | 3 |
| H193Y | 76 | 0.31% | 2 |
| P250L | 76 | 0.31% | 1 |
| V272L | 69 | 0.28% | 3 |
| G154V | 68 | 0.28% | 3 |
| S215I | 45 | 0.18% | 4 |
| V274A | 41 | 0.17% | 0 |
| A138V | 40 | 0.16% | 1 |
| I255F | 40 | 0.16% | 1 |
| V172F | 39 | 0.16% | 1 |
| Total | 2358 | 9.55% | |

^{a}Frequency in 24,679 tumors with mutant p53 missense mutations.
^{b}Mutant tumor cell lines in the IARC TP53 Database.

Activation of the ts mutants alone at 32° C. induced H1299 cell cycle arrest as measured by ³H-thymidine incorporation assay (FIG. 3A), but no apoptosis was detected (FIG. 3B). However, the ts mutants cooperated with CPT to induce apoptosis at 32° C. as indicated by PARP cleavage (FIG. 3B), and loss of cell viability (FIG. 3C, 3D). Apoptotic caspase activation may also explain the decrease of MDM2 in CPT-treated cells (FIG. 2B, 2C), since MDM2 is a substrate for Caspase 2 and 3 (Chen L, et al. J Biol Chem 1997 272:22966-73; Oliver T G, et al. Mol Cell 2011 43:57-71). The results suggest the ts mutants regained some of the tumor suppressor functions at 32° C.

Molecular chaperones mediate ts p53 refolding at low temperature. Time course analysis using R158H and R282W showed that ts p53 induction of p21, MDM2 and PUMA were detectable 4 hours after shifting to 32° C. and peaked after 12-24 hours (FIG. 11A). When the cells were shifted from 32° C. back to 37° C., MDM2 and p21 decreased to background levels in 8-24 hours, whereas PUMA level remained elevated for 24 hours (FIG. 11B). ChIP analysis showed ts p53 DNA binding was activated and peaked 2-4 hours after shifting to 32° C. and was completely inactivated if shifted back to 37° C. for 1 hour, consistent with rapid heat inactivation (FIG. 4A). Inhibition of new protein synthesis by cycloheximide (CHX) did not prevent the rescue of DNA binding at 32° C., indicating that refolding of stockpiled ts p53 contributed to most of the initial activation (FIG. 4B). The hsp90 inhibitor 17-AAG blocked p53 activation at 32° C. (FIG. 4B), suggesting that ts p53 refolding in cells was chaperone-dependent.

To further test whether misfolded ts p53 can post-translationally refold at low temperature, we monitored the refolding using an in vitro system. We recently established a cell-free assay based on luciferase fragment complementation to detect DNA binding by p53-Cluc fusion protein (FIG. 4C) (He F, et al. Proc Natl Acad Sci USA 2019 116:8859-68). R282W-Cluc produced in 16° C. E. coli culture was active in DNA binding at 23° C., but was inactivated after incubating at 34° C.; in contrast, wt p53-Cluc remained active after heating at 34° C. (FIG. 4D). Incubation of heat-inactivated R282W-Cluc at 23° C. for 3 hours did not restore DNA binding. However, co-incubation with a mixture of chaperones (DnaK, DnaJ, ClpB, GrpE, GroESL, FIG. 4E) restored ˜70% of R282W-Cluc DNA binding (FIG. 4D) (de Marco A, et al. BMC Biotechnol 2007 7:32). The chaperones did not rescue the non-ts mutant R175H-Cluc (FIG. 4D). Wt p53-Cluc was modestly resistant to heating at 34° C., its DNA binding was stimulated ˜2-fold by the chaperones (FIG. 4D), suggesting there was refolding of a spontaneously denatured sub-population. The results suggest that post-translational refolding of denatured ts p53 at low temperatures required the assistance of molecular chaperones.

Activation of endogenous ts p53 mutants in tumor cell lines. The IARC TP53 Database showed that ts p53 mutations frequently detected in cancer are also present in many tumor cell lines (Table 3). The correlation suggests the ts mutations in the cell lines were originated from the tumors. Analysis of 7 tumor cell lines expressing ts p53 (6 of the 7 ts mutants were also tested in H1299 cells, FIGS. 2,11) showed increased MDM2, p21, and PUMA expression at 32° C., suggesting the ts p53 mutants were activated in their natural context (FIG. 5A). Cell lines with wt p53 or non-ts p53 mutant were not activated at 320 (FIG. 12A). Knockout of endogenous ts p53 by CRISPR/Cas9 gene editing in 3 cell lines abrogated the induction of MDM2/p21/PUMA and apoptosis at 32° C. (FIGS. 5B,12B,12C), indicating the response was mediated by ts p53. Long incubation of GA10 lymphoma cells at 32° C. led to cell death and population decline (FIG. 5C), whereas GA10-p53KO cells continued to proliferate at 32° C. (slower than 37° C. as expected from less active metabolism). Therefore, endogenous ts p53 can be activated at 32° C. to induce arrest or apoptosis. Analysis of DNA synthesis using ³H-thymidine labeling confirmed cell cycle arrest of lung tumor lines H441 (R158L) and H1355 (E285K) at 32° C. (FIG. 13A). Annexin V staining also showed p53-mediated apoptosis in GA10 (P152L) cells at 32° C. (FIGS. 13B,13C,13D).

Tumors with wt p53 often have silenced ARF expression that result in increased MDM2 activity, whereas tumors with mutant p53 generally retain ARF (Stott F J, et al. Embo J 1998 17:5001-14; Eischen C M, et al. Genes Dev 1999 13:2658-69). The status of ARF in tumors with ts p53 have not been reported. RT-PCR analysis of 7 tumor cell lines with ts p53 showed they expressed various levels of ARF mRNA similar to non-ts mutant or p53-null cells (FIG. 13E). In contrast, 3 cell lines with wt p53 had no detectable ARF mRNA (FIG. 13E). The result suggests that ts p53 mutations also eliminate the selection pressure to silence ARF. The presence of ARF in the ts cell lines should limit MDM2 activity and facilitate p53 activation at 32° C. Consistent with this notion, despite increased MDM2 level at 32° C. there was only modest or no down regulation of p53 in the ts cell lines (FIG. 5A).

Pharmacological induction of hypothermia. To test the effect of hypothermia on tumors with ts p53, the adenosine A1 receptor (A1AR) agonist CHA (N⁶-cyclohexyladenine) was used to lower mouse body temperature to 32° C. for 24-48 hours. Thermal imaging of nude mice in standard ambient condition (23° C.) showed anterior skin temperature of 37-38° C., providing a non-invasive measure of core temperature (FIG. 6A). Intraperitoneal injection of CHA caused body temperature to decrease to ˜3° C. above the environment in ˜10 minutes, consistent with the induction of hibernation-like response in rats and mice by A1AR agonists (Tupone D, et al. J Neurosci 2013 33:14512-25; Futatsuki T, et al. Sci Rep 2018 8:2717). Cell culture test suggested that 24 hours at 32° C. was needed for optimal ts p53 activation (FIG. 11). This was achieved by keeping the CHA-treated mice in a 28° C. environment (FIG. 6A). Using multiple injections of CHA it was possible to maintain body temperature at 32° C. for 24-32 hours. The procedure can be repeated 5-6 times with 2-4 days between treatments to allow recovery (referred to as 24 hr×6 or 32 hr×5 formats).

Hypothermia inhibits the growth of tumors expressing ts p53. To test the effect of ts p53 activation in vivo, nude mice bearing GA10 B-cell lymphoma subcutaneous xenografts were treated with 6 rounds of hypothermia, each round lowering body temperature to ˜32° C. for 24 hours (24 hr×6 format). Tumor growth was attenuated during the treatments, but resumed after the treatments were stopped (FIG. 6B). Untreated 37° C. tumors grew rapidly (FIG. 6B). GA10-p53KO tumors were not inhibited by hypothermia (FIG. 6C), suggesting the effect of hypothermia was mediated by ts p53. Hypothermia also inhibited the growth of H1963 lung tumor xenografts, but did not stop H1963-p53KO tumors (FIG. 6D). In 37° C. control animals (no hypothermia treatment), p53 knockout did not affect the tumor growth rates of GA10/GA10-p53KO and H1963/H1963-p53KO in pair-wise comparisons (FIGS. 14A,14B). The results suggest that hypothermia activated the endogenous ts mutant p53 to inhibit tumor growth.

Hypothermia cooperates with chemotherapy to induce tumor regression. To test whether ts p53 activation cooperates with chemotherapy, large GA10 and GA10-p53KO tumors (˜300-1000 mm³) were treated with hypothermia in combination with CPT. CPT was given at a low dose (1.5 mg/kg) that modestly inhibited GA10 tumor growth at 37° C. but did not cause shrinkage (FIG. 14C). At 37° C. body temperature (p53 inactive), GA10 and GA10-p53KO tumors showed no difference in response to CPT (FIG. 14D). Hypothermia+CPT combination (in 32 hr×5 format) induced significant regression of GA10 tumors (FIGS. 7A,7B,7C). Furthermore, 32% (12/37) of GA10 tumors did not relapse in the 50-day observation period after stopping treatment (FIG. 7C). In contrast, GA10-p53KO tumors did not achieve complete response or durable remission, the p53-independent effects of CPT and hypothermia only led to modest shrinkage (FIG. 7A,7C).

Growth curves of individual tumors with and without relapse showed that durable remissions occurred after achieving >80% shrinkage during the initial treatment (FIG. 7D). Re-treatment of relapsed GA10 tumors resulted in weaker responses compared to the initial cycles, and did not produce new durable remission (FIG. 7E). As expected, GA10-p53KO tumors responded poorly to both initial and repeat treatments (FIG. 15A). Western blot showed PARP cleavage and PUMA expression in GA10 tumors (but not GA10-p53KO) treated for 24 hours with the combination, consistent with ts p53-mediated apoptosis in vivo (FIG. 15). The apoptosis in GA10 tumors was also confirmed by TUNEL staining (FIG. 16). The results showed that ts p53 activation cooperated with chemotherapy to induce tumor regression and durable remission.

### Discussion

P53 inactivation in cancer occurs predominantly by missense mutations that result in accumulation of misfolded protein, providing a tumor-specific target (Leroy B, et al. Hum Mutat 2014 35:672-88; Joerger A C, et al. Annu Rev Biochem 2016 85:375-404). Conformational rescue of mutant p53 has been a long-standing challenge in drug development. Since ˜15% of p53 missense mutants regain activity at 32° C., hypothermia should rescue this subclass of mutants without specific drugs. Physical cooling should activate ts p53 uniformly, bypassing common limitations in drug delivery. Furthermore, hypothermia does not damage normal tissues, whereas small molecules often have off-target toxicity. The results described in this study demonstrate that ts mutant p53 can be activated in tumors by inducing whole-body hypothermia, resulting in synergistic anti-tumor effects when combined with chemotherapy. The finding raises the possibility of repurposing therapeutic hypothermia for tumors with ts p53 mutations.

Tumor development selects for p53 mutations that inactivate DNA binding. Apparently many ts mutants are also selected because they are inactive at 37° C. The p53 ts mutations analyzed in the current work were defined by their re-activation at 32° C. in yeast and mammalian cell culture (Shiraishi K, et al. J Biol Chem 2004 279:348-55). Therefore, the animal hypothermia experiments were also performed at 32° C. in order to balance refolding efficiency, cellular metabolic activity, and clinical relevance. Analysis of 17 frequently observed p53 ts mutants (affecting ˜64% of tumors expressing ts p53) showed they regained similar ability to induce target genes and growth arrest at 32° C., suggesting that similar results can be expected for other ts mutants.

Wt p53 is tightly regulated by the MDM2 feedback loop. A subset of tumors bypass the need to mutate p53 by silencing ARF or overexpressing MDM2 (Stott F J, et al. Embo J 1998 17:5001-14; Eischen C M, et al. Genes Dev 1999 13:2658-69). Tumors with mutant p53 typically accumulate the p53 protein to high levels, partly due to inability to induce MDM2 and oncogene-activated expression of ARF. Activating ts p53 should in principle trigger its own degradation by MDM2. However, analysis of tumor cell lines expressing endogenous ts p53 showed no significant degradation at 32° C. despite induction of MDM2. Ts p53 tumor cell lines retained ARF mRNA expression similar to non-ts mutant cell lines, possibly played a role in preventing p53 degradation at 32° C. and sensitizing them to the cell cycle arrest or apoptotic effects of rescued p53.

If tumor development selects for complete loss of p53 activity, tissues that are not constantly maintained at 37° C. (such as skin and breast) should have lower frequency of ts p53 mutations since they will be partially active and have less advantage. The COSMIC database shows that breast tumors have below-average ts p53 mutation frequency as expected, but surprisingly skin cancer has above-average frequency (Table 2). The reason for the discrepancy remains to be determined. It is possible that exposure to specific mutagens (i.e., UV irradiation) distorted the ts p53 mutation frequency in skin cancer. Alternatively, skin cancer may have more frequent ARF silencing or MDM2 overexpression that neutralize residual ts p53 activity at below −37° C. temperatures.

Most ts p53 mutations are located at the hydrophobic p sandwich core of the DNA binding domain that reduce thermo stability (Joerger A C, et al. Proc Natl Acad Sci USA 2006 103:15056-61). Our results suggest that ts p53 exists in a dynamic equilibrium between misfolded and wt conformations in the cell. Post-translational refolding of mutant protein contributes to rapid restoration of activity at 32° C. Furthermore, molecular chaperones are essential for rapid refolding at permissive temperature. Cell-free analysis showed that ts p53 cannot spontaneously regain wt conformation at low temperature without the help of molecular chaperones. The multi-domain organization and tetrameric status of p53 may lead to dependence on chaperones for accurate refolding. Previous work showed that optimal wt p53 activity in cells at 37° C. required hsp90 (Walerych D, et al. J Biol Chem 2004 279:48836-45). Hsp40/Hsp70 and Hsp90 exert opposing activities on the p53 DNA binding domain to maintain its conformation at a dynamic equilibrium (Boysen M, et al. Mol Cell 2019 74:831-43 e4; Dahiya V, et al. Mol Cell 2019 74:816-30 e7).

Our results demonstrated that hypothermia can induce rapid regression of lymphomas and frequently achieved durable remission. Therefore, its potential in treating tumors with ts p53 warrants further investigation. The hypothermia procedure currently used in the clinic is performed under intensive care setting. Repurposing this procedure for cancer will require significant therapeutic benefits. Development of non-invasive methods to induce hypothermia will lower the threshold for translation to cancer treatment. Recent work identified specific neurons in the mouse hypothalamus that are sufficient to induce hypothermia upon stimulation (Takahashi™, et al. Nature 2020 583:109-14; Hrvatin S, et al. Nature 2020 583:115-21). Hypothermia induction by anti-psychotic drugs is also well-documented (Tarahovsky Y S, et al. Psychopharmacology (Berl) 2017 234:173-84). It is important to note that hypothermia affects energy metabolism, pharmacodynamics, immune functions, and physiology. Further studies will be needed to translate this strategy for cancer therapy.

### Example 2: Targeting Tumors by Local Cooling

Tumors such as intraperitoneal and bladder tumors are potential candidates for local hypothermia treatment. Both tumors are treated with regional hyperthermic chemotherapy (42° C. for 1-2 hrs) by irrigation with heated drug solution, and should also be accessible by 32° C. hypothermic irrigation. Non-muscle invasive bladder cancer (NMIBC) is uniquely suitable for this approach because the superficial location of NMIBC and the availability of bladder irrigation device (Synergo) with cooling function. P53 mutations are prevalent (˜70%) in high-grade non-muscle invasive bladder cancer. Analysis of bladder cancer p53 missense mutations in the COSMIC database showed a 25% (115/465) TS mutation frequency. Three bladder cancer cell lines were validated with TS mutant p53 (FIG. 17A), confirming their induction of p53 target genes (MDM2 and p21) at 32° C.

Standard bladder intravesical chemotherapy involves 1-2 hr irrigation with drugs at 37° C. or 42° C. The effect of treating cells with 32° C. hypothermia after chemotherapy was tested to avoid interference with drug uptake. TS mutant p53 activation peaks 6-8 hrs after shift to 32° C., which should cooperate with chemotherapy-induced DNA damage to induce cell death. Experiments using the UC3 bladder cancer cells showed that after 2 hrs drug treatment and washout, incubating the treated cells at 32° C. for 24 hrs significantly increased the growth inhibition potency of Camptothecin, and modestly increased the effects of Doxorubicin and Gemcitabine (FIG. 17B). The result suggests that hypothermia treatment can be performed after chemotherapy to enhance response, without altering the standard chemotherapy procedure. Saline irrigation of up to 24 hrs is routinely performed after transurethral resection of bladder tumor (TURBT) procedure to prevent clotting. Therefore, irrigation with cooled saline after intravesical chemotherapy is expected to be well-tolerated and safe.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating a tumor having a temperature sensitive p53 (ts p53) mutation in a subject in need thereof, comprising
(a) inducing moderate hypothermia in the tumor for a duration sufficient to activate the mutant p53, and
(b) administering to the subject a chemotherapeutic drug.

2. The method of claim 1, wherein hypothermia is induced by administering to the subject an effective amount of an anti-psychotic drug.

3. The method of claim 2, wherein the anti-psychotic drug is selected from the group consisting of Zuclopenthixol, Flupenthixol, Chlorprothixen, Tiotixene, Clopenthixol, Thioridazine, Chlorpromazine, Levomepromazine, Cyamemazine, Periciazine, Pipothiazine, Fluphenazine, Trifluoperazine, Perphenazine, Prochlorperazine, Promazine, Mesoridazine, Haloperidol, Pipamperone, Droperidol, Benperidol, Tiapride, Sulpiride, Amisulpiride, Sultopride, Loxapine, Pimozide, Zotepine, Prothipendyl, Penfluridol, Risperidone, Clozapine, Olanzapine, Quetiapine, Aripiprazole, and Ziprasidone.

4. The method of claim 1, wherein hypothermia is induced with core and/or peripheral cooling of the subject, with or without anesthesia.

5. The method of claim 1, wherein the tumor temperature is maintained at a temperature of about 32° C. for 24 to 48 hours.

6. The method of claim 1, further comprising assaying a sample from the subject for a ts p53 mutation.

7. The method of claim 6, wherein the ts p53 mutation is an amino acid substitution selected from the group consisting of S99, F113, Y126, N131, C135, A138, V143, W146, P151, P152, G154, T155, R156, R158, M160, A161, Q165, V172, R175, A189, P190, H193, R196, V197, Y205, D208, T211, F212, R213, H214, S215, V216, V217, P219, P223, C229, T231, I232, Y234, S240, M246, N247, R249, P250, I251, L252, T253, I254, I255, T256, L257, N268, F270, E271, V272, V274, R282, R283, E285, and E286, or any combination thereof.

8. The method of claim 7, wherein the ts p53 mutation is selected from the group consisting of S99F, F113C, Y126C, N131H, C135W, A138V, V143A, W146G, P151A, P152L, G154V, T155I, R156G, R158H, M160I, A161G, Q165L, V172F, R175G, A189P, P190T, H193Y, R196L, V197L, Y205N, D208A, T211A, F212V, R213G, H214R, S215I, V216L, V217G, P219L, P223A, C229G, T231A, I232F, Y234C, S240R, M246V, N247I, R249K, P250L, I251L, L252F, T253A, I254F, I255F, T256A, L257V, N268I, F270I, E271K, V272M, V274A, R282W, R283H, E285K, and E286G, or any combination thereof.

9. The method of claim 1, wherein the tumor is an intraperitoneal tumor.

10. The method of claim 1, wherein the tumor is a bladder tumor.

11. The method of claim 9, wherein the bladder is cooled by circulating cooled buffer through a catheter in the bladder.

12. The method of claim 1, wherein the moderate hypothermia is induced simultaneously with administration of the chemotherapeutic drug.

13. The method of claim 1, wherein the moderate hypothermia is induced 1 minute to 1 day after the chemotherapeutic drug is administered.
